(19) United States
(12) Patent Application Publication
(10) Pub. No.: US 2017/0362195 A1
(43) Pub. Date: Dec. 21, 2017

(54) METHODS FOR THE MANUFACTURE OF CANNABINOID PRODRUGS, PHARMACEUTICAL FORMULATIONS AND THEIR USE

(71) Applicant: Full Spectrum Laboratories Ltd, Dublin (IE)

(72) Inventors: Richard C. Peet, Tampa, FL (US); Malcolm J. Kavarana, Tampa, FL (US)

(21) Appl. No.: 15/625,620

(22) Filed: Jun. 16, 2017

Related U.S. Application Data
(60) Provisional application No. 62/351,103, filed on Jun. 16, 2016.

(51) Int. Cl. C07D 311/80; C07C 235/74; C07C 229/08; C12P 17/06; C12P 13/08

(52) U.S. Cl. CPC C07D 311/80; C12P 17/06; C12P 13/08; C12Y 121/03007; C07C 229/08; C12Y 121/03008; C07C 235/74

(57) ABSTRACT

Described are methods for producing cannabinoid prodrugs as well as methods for formulating such prodrugs in a pharmaceutically acceptable form and their use as therapeutic agents for treating diseases.

This application claims the benefit of priority of U.S. Provisional Patent Application No. 62/351,103 that was filed on Jun. 16, 2016.

### FIELD OF THE INVENTION

The present invention relates to methods for the manufacture of cannabinoid prodrugs. Specifically, the present invention relates to enzyme-catalyzed synthesis of cannabinoid prodrugs as well as to methods for manufacturing cannabinoid prodrugs by chemical modification of a cannabinoid or a cannabinoid compound synthesized chemically, bio-catalytically, or by using synthetic biology.

### BACKGROUND OF THE INVENTION

Cannabinoids are terpenophenolic compounds found in Cannabis saliva, an annual plant belonging to the Cannabaceae family. The plant contains more than 400 chemicals and approximately 70 cannabinoids, which accumulate mainly in the glandular trichomes. The main psychoactive cannabinoid is tetrahydrocannabinol (THC) or more precisely its main isomer (−)-trans-Δ⁹-tetrahydrocannabinol ((6aR,10aR)-Δ⁹-tetrahydrocannabinol), which is used for treating a wide range of medical conditions, including glaucoma, AIDS wasting, neuropathic pain, treatment of spasticity associated with multiple sclerosis, fibromyalgia and chemotherapy-induced nausea. THC is also effective for treating allergies, inflammation, infection, epilepsy, depression, migraine, bipolar disorders, anxiety disorder, drug dependency and drug withdrawal syndromes.

In addition to THC, other biologically active cannabinoids are also present in C. sativa plant. One such cannabinoid is cannabidiol (CBD), an isomer of THC, which is a potent antioxidant and anti-inflammatory compound known to provide protection against acute and chronic neuro-degeneration. Another biologically active cannabinoid is cannabigerol (CBG). CBG is found in high concentrations in hemp. It is a high affinity α₂-adrenergic receptor agonist, a moderate affinity 5-HT_{1A} receptor antagonist and is a low affinity CB1 receptor antagonist. CBG is known to possess a mild anti-depressant activity. Cannabichromene (CBC) is another biologically active cannabinoid and is known to possess anti-inflammatory, anti-fungal and anti-viral properties.

This application describes the use of synthetic biology and bio-catalysis to manufacture pharmaceutical grade cannabinoid therapeutics. More specifically, this application describes methods for the enzyme catalyzed synthesis of pharmaceutically acceptable prodrugs cannabinoid analog.

### SUMMARY OF THE INVENTION

The present invention provides a method for producing a cannabinoid prodrug according to Formula Ia or Formula IIa:

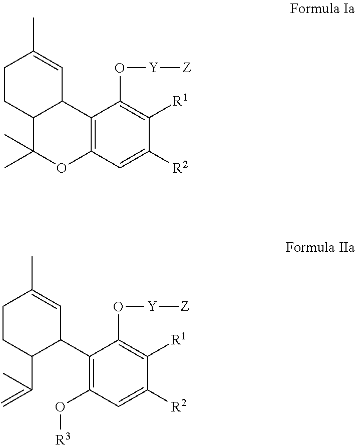

According to the disclosed method, Formula Ia and Formula IIa compounds are synthesized by contacting a compound according to Formula I or Formula II

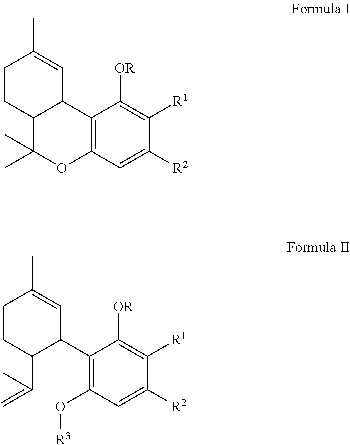

with an activated —Y—Z reagent. For Formula I or Formula II compounds R is —H, substituent R¹ is —H, —COOH, or —COO(C₁-C₅)alkyl, R² is a group selected from (C₁-C₁₀)alkyl, (C₂-C₁₀)alkenyl, (C₂-C₁₀)alkynyl, (C₃-C₁₀)cycloalkyl, (C₃-C₁₀)cycloalkylalkylene, (C₃-C₁₀)aryl, and (C₃-C₁₀)arylalkylene, and substituent R³ is —H, or (C₁-C₅)alkyl.

For cannabinoid compounds according to the inventive method, —Z is selected from the group consisting of -hemisuccinate, -succinate, -oxalate, —C(O)—CH₂—[OCH₂CH₂]ₙ—OR⁴, —C(O)—CH₂—[OCH₂CH₂]ₙ—NH₂, —C(O)[CH₂]ₙ—NR⁴R⁵, —C(O)O[CH₂]ₙ—NR⁴R⁵, —C(O)—NH—[CH₂]ₙ—NR⁴R⁵, —C(O)[CH₂]ₙ—N⁺(R⁴)(R⁵))(R⁶)X⁻, —C(O)O[CH₂]ₙ—N⁺(R⁴)(R⁵)(R⁶)X⁻, —C(O)—NH—[CH₂]ₙ—N⁺(R⁴)(R⁵))(R⁶)X⁻; or an -oligosaccharide. Alternatively, —Y—Z is an oligosaccharide.

Variable “Y” is a group selected from L-amino acid residue, a D-amino acid residue, a β-amino acid residue, a γ-amino acid residue, —C(O)—CH₂—[OCH₂CH₂]ₙ—O—, and —C(O)—CH₂—[OCH₂CH₂]ₙ—NH—, while substituents R⁴, R⁵, and R⁶ are each independently selected from the group consisting of —H, —OH, formyl, acetyl, pivaloyl, and (C₁-C₅)alkyl.

For cannabinoid compounds of the invention, subscript “n” is an integer, such as 1, 2, 3, 4, 5, or 6, while “X” is a counter ion derived from a pharmaceutically acceptable acid.

In one embodiment, Formula I or Formula II compounds are obtained by contacting a compound of Formula III

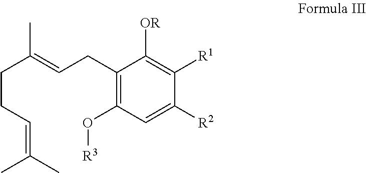

with a cannabinoid synthase, selected from the group consisting of tetrahydrocannabivarin acid synthase (THCVA synthase), tetrahydrocannabinolic acid synthase (THCA synthase), cannabidiolic acid synthase (CBDA synthase), and cannabichromene acid synthase (CBCA synthase).

For Formula III compounds substituents R, R¹, R², and R³ are as defined above. In one embodiment, the compound of Formula III is contacted with the cannabinoid synthase in the presence of a solvent selected from the group consisting of water, phosphate buffer, citrate buffer, TRIS buffer, HEPES buffer, a mixture of water and a (C₁-C₅)alcohol, and a mixture of buffer and a (C₁-C₅)alcohol.

According to one embodiment, —Z is -hemisuccinate, -succinate, —C(O)—CH₂—[OCH₂CH₂]ₙ—OR⁴, or —C(O)—CH₂—[OCH₂CH₂]ₙ—NH₂.

For certain Formula I and Formula II compounds, —Z is —C(O)—CH₂—[OCH₂CH₂]ₙ—OR⁴. In one exemplary embodiment —Y is valine and —Y—Z is -valine-C(O)—CH₂—[OCH₂CH₂]ₙ—OR⁴. For such cannabinoid compounds substituent R⁴ is —H or methyl, and subscript “n” is 1, 2, 3, or 4.

In one embodiment, —Y—Z is a —Y-oligosaccharide. Illustrative “Y” groups include without limitation —C(O)—CH₂—[OCH₂CH₂]ₙ—O—, —C(O)—CH₂—[OCH₂CH₂]ₙ—OCH₂CH₂C(O)—, a polyethylene glycol moiety as well as a L-amino acid residue, a D-amino acid residue, a β-amino acid residue, or a γ-amino acid residue.

In one embodiment, R¹ is —COOH, and R² is (C₁-C₁₀)alkyl for compounds according to the claimed method, for example, R² is propyl or pentyl.

According to an embodiment of the invention, when R¹ is —COOH, the cannabinoid compound can be optionally de-carboxylated by heating a solution of the Formula I, Ia, II or IIa cannabinoid compound, or exposing a solution of the Formula I, Ia, II or IIa cannabinoid compound to UV-light.

Encompassed by another embodiment, is a method for producing a cannabinoid prodrug of Formula IVa or Formula Va:

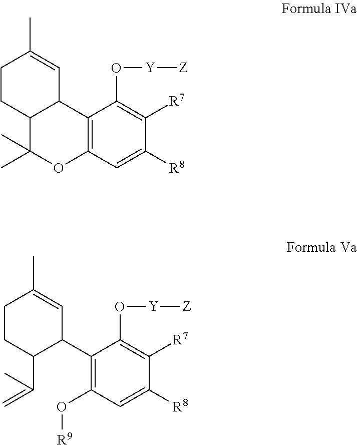

Such cannabinoid prodrugs are produced by (a) contacting a compound of Formula VI:

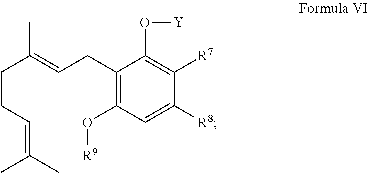

with a cannabinoid synthase to obtain a compound according to Formula IV or Formula V:

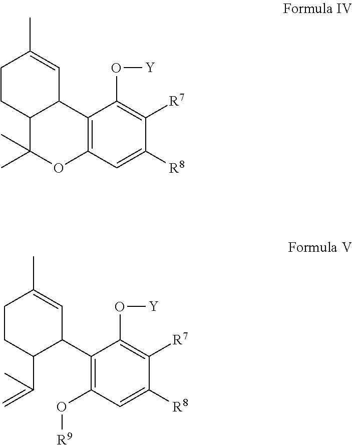

According to the inventive method, Formula IV or Formula V compounds are contacted with an activated —Z reagent to obtain the Formula IVa and Formula Va compounds. For Formula IVa or Formula Va compounds, substituent R⁷ is —H, —COOH, or —COO(C₁-C₅)alkyl, R⁸ is a group selected from (C₁-C₁₀)alkyl, (C₂-C₁₀)alkenyl, (C₂-C₁₀)alkynyl, (C₃-C₁₀)cycloalkyl, (C₃-C₁₀)cycloalkylalkylene, (C₃-C₁₀)aryl, and (C₃-C₁₀)arylalkylene, and substituent R⁹ is —H, or (C₁-C₅)alkyl.

Variable “Y” in Formulae IV, V, VI, IVa and Formula Va is a group selected from L-amino acid residue, a D-amino acid residue, a β-amino acid residue, a γ-amino acid residue, —C(O)—CH₂—[OCH₂CH₂]ₙ—OCH₂CH₂C(O)—, and —C(O)—CH₂—[OCH₂CH₂]ₙ—NH—, while variable Z is selected from the group consisting of hemisuccinate, succinate, oxalate, —C(O)—CH₂—[OCH₂CH₂]ₙ—OR¹⁰, —C(O)—CH₂—[OCH₂CH₂]ₙ—NH₂, —C(O)[CH₂]ₙ—NR¹⁰R¹¹, —C(O)O[CH₂]ₙ—NR¹⁰R¹¹, —C(O)—NH—[CH₂]ₙ—NR¹⁰R¹¹, —C(O)[CH₂]ₙ—N⁺(R¹⁰)(R¹¹)(R¹²)X⁻, —C(O)O[CH₂]ₙ—N⁺(R¹⁰)(R¹¹)(R¹²)X⁻, and —C(O)—NH—[CH₂]ₙ—N⁺(R¹⁰)(R¹¹))(R¹²)X⁻.

For Formula IVa and Formula Va compounds substituents R¹⁰, R¹¹, and R¹² are each independently selected from the group consisting of —H, —OH, formyl, acetyl, pivaloyl, and (C₁-C₅)alkyl, subscript “n” is 1, 2, 3, 4, 5, or 6; and “X” is a counter ion derived from a pharmaceutically acceptable acid.

In yet another embodiment, the disclosure provides a method for producing a cannabinoid prodrug of Formula VIIa or Formula VIIIa:

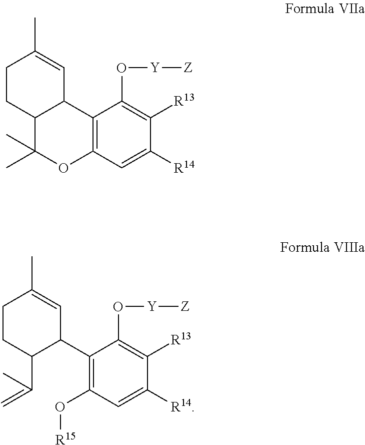

According to the inventive method, the Formula VIIa and VIIIa compounds are obtained by (a) contacting a compound of Formula IX with a cannabinoid synthase.

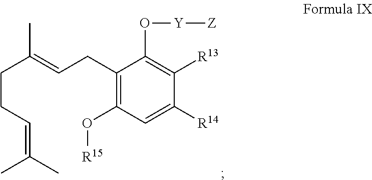

For Formula VIIa and VIIIa compounds, R¹³ is —H, —COOH, or —COO(C₁-C₅)alkyl, R¹⁴ is selected from the group consisting of (C₁-C₁₀)alkyl, (C₂-C₁₀)alkenyl, (C₂-C₁₀)alkynyl, (C₃-C₁₀)cycloalkyl, (C₃-C₁₀)cycloalkylalkylene, (C₃-C₁₀)aryl, and (C₃-C₁₀)arylalkylene, and substituent R¹⁵ is —H, or (C₁-C₅)alkyl.

Variable “Y” in —Y—Z is selected from L-amino acid residue, a D-amino acid residue, a β-amino acid residue, a γ-amino acid residue, —C(O)—CH₂—[OCH₂CH₂]ₙ—O—, and —C(O)—CH₂—[OCH₂CH₂]ₙ—NH—, while variable “Z” is group selected from succinic anhydride, hemisuccinate, succinate, oxalate, —C(O)—CH₂—[OCH₂CH₂]ₙ—OR⁴, —C(O)—CH₂—[OCH₂CH₂]ₙ—NH₂, —C(O)[CH₂]ₙ—NR¹⁶R¹⁷, —C(O)O[CH₂]ₙ—NR¹⁶R¹⁷, —C(O)—NH—[CH₂]ₙ—NR¹⁶R¹⁷, —C(O)[CH₂]ₙ—N⁺(R¹⁶)(R¹⁷))(R¹⁸)X⁻, —C(O)O[CH₂]ₙ—N⁺(R¹⁶)(R¹⁷)(R¹⁸)X⁻, and —C(O)—NH—[CH₂]ₙ—N⁺(R¹⁶)(R¹⁷))(R¹⁸)X⁻.

For Formula VIIa and VIIIa compounds, substituents R¹⁶, R¹⁷, and R¹⁸ are each independently selected from the group consisting of —H, —OH, formyl, acetyl, pivaloyl, and (C₁-C₅)alkyl, subscript “n” is 1, 2, 3, 4, 5, or 6; and “X” is a counter ion derived from a pharmaceutically acceptable acid.

### DETAILED DESCRIPTION

### Definitions

As used herein, unless otherwise stated, the singular forms “a,” “an,” and “the” include plural reference. Thus, for example, a reference to “a cell” includes a plurality of cells, and a reference to “a molecule” is a reference to one or more molecules.

As used herein, “about” will be understood by persons of ordinary skill in the art and will vary to some extent depending upon the context in which it is used. If there are uses of the turn which are not clear to persons of ordinary skill in the art, given the context in which it is used, “about” will mean up to plus or minus 10% of the particular term.

The term “alkyl” refers to a straight or branched chain, saturated hydrocarbon having the indicated number of carbon atoms. For example, (C₁-C₁₀)alkyl is meant to include but is not limited to methyl, ethyl, propyl, isopropyl, butyl, sec-butyl, tert-butyl, pentyl, isopentyl, neopentyl, hexyl, isohexyl, and neohexyl, etc. An alkyl group can be unsubstituted or optionally substituted with one or more substituents as described herein below.

The term “alkenyl” refers to a straight or branched chain unsaturated hydrocarbon having the indicated number of carbon atoms and at least one double bond. Examples of a (C₂-C₁₀)alkenyl group include, but are not limited to, ethylene, propylene, 1-butylene, 2-butene, isobutene, sec-butene, 1-pentene. 2-pentene, isopentene, 1-hexene, 2-hexene, 3-hexene, isohexene, 1-heptene, 2-heptene, 3-heptene, isoheptene, 1-octene, 2-octene, 3-octene, 4-octene, and isooctene. An alkenyl group can be unsubstituted or optionally substituted with one or more substituents as described herein below.

The term “alkynyl” refers to a straight or branched chain unsaturated hydrocarbon having the indicated number of carbon atoms and at least one triple bond. Examples of a (C₂-C₁₀)alkynyl group include, but are not limited to, acetylene, propyne, 1-butyne, 2-butyne, 1-pentyne, 2-pentyne, 1-hexyne, 2-hexyne, 3-hexyne, 1-heptyne, 2-heptyne, 3-heptyne, 1-octyne, 2-octyne, 3-octyne and 4-octyne. An alkynyl group can be unsubstituted or optionally substituted with one or more substituents as described herein below.

The term “alkoxy” refers to an —O-alkyl group having the indicated number of carbon atoms. For example, a (C₁-C₆)alkoxy group includes —O-methyl, —O-ethyl, —O-propyl, —O-isopropyl, —O-butyl, —O-sec-butyl, —O-tert-butyl, —O-pentyl, —O-isopentyl, —O-neopentyl, —O-hexyl, —O-isohexyl, and —O-neohexyl.

The term “aryl” refers to a 3- to 14-member monocyclic, bicyclic, tricyclic, or polycyclic aromatic hydrocarbon ring system. Examples of an aryl group include naphthyl, pyrenyl, and anthracyl. An aryl group can be unsubstituted or optionally substituted with one or more substituents as described herein below.

The terms “alkylene,” “cycloalkylene,” “alkenylene,” “alkynylene,” “arylene,” and “heteroarylene,” alone or as part of another substituent, means a divalent radical derived from an alkyl, cycloalkyl, alkenyl, alkynyl, aryl, or heteroaryl group, respectively, as exemplified by —CH₂CH₂CH₂CH₂—. For alkylene, alkenylene, or aryl linking groups, no orientation of the linking group is implied.

The term “halogen” and “halo” refers to —F, —Cl, —Br or —I.

The term “heteroatom” is meant to include oxygen (O), nitrogen (N), and sulfur (S).

A “hydroxyl” or “hydroxy” refers to an —OH group.

The term “hydroxyalkyl,” refers to an alkyl group having the indicated number of carbon atoms wherein one or more of the alkyl group's hydrogen atoms is replaced with an —OH group. Examples of hydroxyalkyl groups include, but are not limited to, —CH₂OH, —CH₂CH₂OH, —CH₂CH₂CH₂OH, —CH₂CH₂CH₂CH₂OH, —CH₂CH₂CH₂CH₂CH₂OH, —CH₂CH₂CH₂CH₂CH₂CH₂OH, and branched versions thereof.

The term “cycloalkyl” or “carbocycle” refer to monocyclic, bicyclic, tricyclic, or polycyclic, 3- to 14-membered ring systems, which are either saturated, unsaturated or aromatic. The heterocycle may be attached via any heteroatom or carbon atom. Cycloalkyl include aryls and hetroaryls as defined above. Representative examples of cycloalky include, but are not limited to, cycloethyl, cyclopropyl, cycloisopropyl, cyclobutyl, cyclopentyl, cyclohexyl, cyclopropene, cyclobutene, cyclopentene, cyclohexene, phenyl, naphthyl, anthracyl, benzofuranyl, and benzothiophenyl. A cycloalkyl group can be unsubstituted or optionally substituted with one or more substituents as described herein below.

The term ‘nitrite or cyano” can he used interchangeably and refer to a —CN group which is bound to a carbon atom of a heteroaryl ring, aryl ring and a heterocycloalkyl ring.

The term “amine or amino” refers to an —NR_{c}R_{d} group wherein R_{c} and R_{d} each independently refer to a hydrogen, (C₁-C₈)alkyl, aryl, heteroaryl, heterocycloalkyl, (C₁-C₈)haloalkyl, and (C₁-C₆)hydroxyalkyl group.

The term “alkylaryl” refers to C₁-C₈ alkyl group in which at least one hydrogen atom of the C₁-C₈ alkyl chain is replaced by an aryl atom, which may be optionally substituted with one or more substituents as described herein below. Examples of alkylaryl groups include, but are not limited to, methylphenyl, ethylnaphthyl, propylphenyl, and butylphenyl groups.

“Arylalkylene” refers to a divalent alkylene wherein one or more hydrogen atoms in the C₁-C₁₀ alkylene group is replaced by a (C₃-C₁₄)aryl group. Examples of (C₃-C₁₄)aryl-(C₁-C₁₀)alkylene groups include without limitation 1-phenylbutylene, phenyl-2-butylene, 1-phenyl-2-methylpropylene, phenylmethylene, phenylpropylene, and naphthylethylene.

“Arylalkenylene” refers to a divalent alkenylene wherein one or more hydrogen atoms in the C₂-C₁₀ alkenylene group is replaced by a (C₃-C₁₄)aryl group.

The term “arylalkynylene” refers to a divalent alkynylene wherein one or more hydrogen atoms in the C₂-C₁₀ alkynylene group is replaced by a (C₃-C₁₄)aryl group.

The terms “carboxyl” and “carboxylate” include such moieties as may be represented by the general formulas:

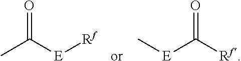

E in the formula is a bond or O and R^{f} individually is H, alkyl, alkenyl, aryl, or a pharmaceutically acceptable salt. Where E is O, and R^{f} is as defined above, the moiety is referred to herein as a carboxyl group, and particularly when R^{f} is a hydrogen, the formula represents a “carboxylic acid”. In general, where the expressly shown oxygen is replaced by sulfur, the formula represents a “thiocarbonyl” group.

Unless otherwise indicated, “stereoisomer” means one stereoisomer of a compound that is substantially free of other stereoisomers of that compound. Thus, a stereomerically pure compound having one chiral center will be substantially free of the opposite enantiomer of the compound. A stereomerically pure compound having two chiral centers will be substantially free of other diastereomers of the compound. A typical stereomerically pure compound comprises greater than about 80% by weight of one stereoisomer of the compound and less than about 20% by weight of other stereoisomers of the compound, for example greater than about 90% by weight of one stereoisomer of the compound and less than about 10% by weight of the other stereoisomers of the compound, or greater than about 95% by weight of one stereoisomer of the compound and less than about 5% by weight of the other stereoisomers of the compound, or greater than about 97% by weight of one stereoisomer of the compound and less than about 3% by weight of the other stereoisomers of the compound.

If there is a discrepancy between a depicted structure and a name Oven that structure, then the depicted structure controls. Additionally, if the stereochemistry of a structure or a portion of a structure is not indicated with, for example, bold or dashed lines, the structure or portion of the structure is to be interpreted as encompassing all stereoisomers of it.

The present invention focuses on biosynthetic methodologies for the manufacture of a prodrug of a cannabinoid. More specifically, the invention relates to enzyme-catalyzed synthesis of a prodrug of a cannabinoid.

The terms “activated reagent” and “active reagent” are used interchangeably and denote a first compound or chemical moiety having one or more functional groups that are together or independently activated prior to contacting such a first compound or chemical moiety with a second compound or chemical moiety to form a covalent bond. Exemplary activated forms of a carboxylic acid include acid halides, acid anhydrides, alkyl esters, and aryl esters. Activation of carboxylic acids and their related coupling chemistries are well known in the chemical and peptide arts. In some instances, a first compound having a carboxylic acid or an activated form of a carboxylic acid couples to a second compound having an amine or hydroxyl group using one or more coupling reagents. Illustrative coupling reagents include carbodiimides such as dicyclohexylcarbodiimide (DCC), ethyl-(Nl,N′-dimethylamino)propylcarbodiimide hydrochloride (EDC), and diisopropylcarbodiimide (DIC). Additional examples include benzotriazol-1-yloxy)tris(dimethylamino)phosphonium hexafluorophosphate (BOP), benzotriazol-1-yloxy)tripyrrolidinophosphonium hexafluorophosphate, (7-Azabenzotriazol-1-yloxy)tripyrrolidinophosphonium hexafluorophosphate (PyAOP), bromotripyrrolidinophosphonium hexafluorophosphate, O-(Benzotriazol-1-yl)-N,N,N′,N′-tetramethyluronium hexafluorophosphate (HBTU) and O-(Benzotriazol-1-yl)-N,N,N′,N′-tetramethyluronium tetrafluoroborate (TBTU), O-(7-Azabenzotriazol-1-yl)-N,N,N′,N′-tetramethyluronium hexafluorophosphate (HATU), O-(7-Azabenzotriazol-1-yl)-N,N,N′,N′-tetramethyluronium tetrafluoroborate (TATU), and O-(6-Chlorobenzotriazol-1-yl)-N,N,N′,N′-tetramethyluronium hexafluorophosphate (HCTU), or any other coupling reagent known in the chemical and peptide arts.

The term “prodrug” refers to a precursor of a biologically active pharmaceutical agent (drug). Prodrugs must undergo a chemical or a metabolic conversion to become a biologically active pharmaceutical agent. A prodrug can be converted ex vivo to the biologically active pharmaceutical agent by chemical transformative processes. In vivo, a prodrug is converted to the biologically active pharmaceutical agent by the action of a metabolic process, an enzymatic process or a degradative process that removes the prodrug moiety to form the biologically active pharmaceutical agent.

In one embodiment, the inventive disclosure provides a method of producing a prodrug of a cannabinoid compound or a prodrug of a cannabis compound by chemically modifying a cannabinoid compound to its prodrug using synthons for such prodrugs. In the context of this disclosure, the terms “cannabinoid compound” and “cannabis compound” are synonymous and used interchangeably to refer to a natural phytocannabinoid, or a cannabinoid synthesized chemically, bioenzymatically, using synthetic biology, or through a combination of chemical and bio-enzymatic processes.

In the context of the present invention the term “analog” refers to a compound that is structurally related to naturally occurring cannabinoids, but whose chemical and biological properties may differ from naturally occurring cannabinoids. In the present context, analog or analogs refer compounds that may not exhibit one or more unwanted side effects of a naturally occurring cannabinoid. Analog also refers to a compound that is derived from a naturally occurring cannabinoid by chemical, biological or a semi-synthetic transformation of the naturally occurring cannabinoid.

Thus, the invention provides a method for making a Formula Ia or a Formula IIa prodrug,

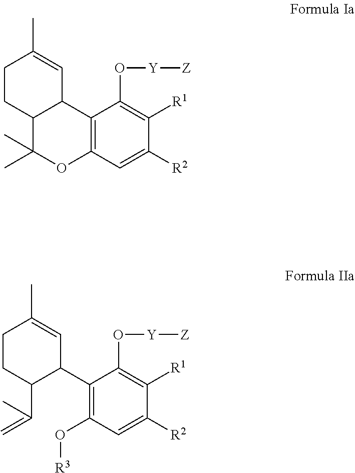

The inventive prodrugs are produced by contacting a compound according to Formula I or Formula II:

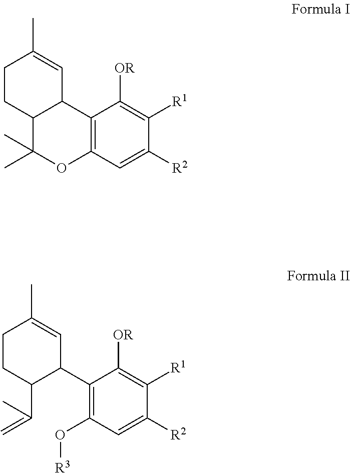

with an activated —Y—Z reagent For Formula I and II compounds, R is —H, R¹ is —H, —COOH, or —COO(C₁-C₅)alkyl, R² is selected from the group consisting of (C₁-C₁₀)alkyl, (C₂-C₁₀)alkenyl, (C₂-C₁₀)alkynyl, (C₃-C₁₀)cycloalkyl, (C₃-C₁₀)cycloalkylalkylene, (C₃-C₁₀)aryl, and (C₃-C₁₀)arylalkylene, and R³ is —H, or (C₁-C₅)alkyl.

In one embodiment, R¹ is —COOH and R² is a (C₁-C₁₀)alkyl, for example, a methyl, ethyl, propyl, iso-propyl, butyl, sec-butyl, iso-butyl, t-butyl, pentyl or hexyl.

According to one embodiment, inventive prodrugs with a carboxylic acid (—COOH) group as the R¹ substituent can undergo an optional decarboxylation step prior to their use as pharmaceutical or nutraceutical agents.

In one embodiment, R¹ is —COOH and R² is pentyl and “Z” in —Y—Z is a group selected from hemisuccinate, -succinate, -oxalate, —C(O)—CH₂—[OCH₂CH₂]ₙ—OR⁴, —C(O)—CH₂—[OCH₂CH₂]ₙ—NH₂, —C(O)[CH₂]ₙ—NR⁴R⁵, —C(O)O[CH₂]ₙ—NR⁴R⁵, —C(O)—NH—[CH₂]ₙ—NR⁴R⁵, —C(O)[CH₂]ₙ—N⁺(R⁴)(R⁵))(R⁶)X⁻, —C(O)O[CH₂]ₙ—N⁺(R⁴)(R⁵)(R⁶)X⁻, and —C(O)—NH—[CH₂]ₙ—N⁺(R⁴)R⁵))(R⁶)X⁻, or an -oligosaccharide. Alternatively, for some Formula Ia or Formula IIa compounds —Y—Z is an oligosaccharide.

For certain cannabinoid prodrugs of the invention, “—Z” is -hemisuccinate, -succinate, —C(O)—CH₂—[OCH₂CH₂]ₙ—OR⁴, or —C(O)—CH₂—[OCH₂CH₂]ₙ—NH₂.

In one embodiment the cannabinoid prodrug is a compound in which, R¹ is —COOH and R² is pentyl and —Z is -hemisuccinate.

In one embodiment the cannabinoid prodrug is a compound in which, R¹ is —COOH and R² is pentyl and —Z is —C(O)—CH₂—[OCH₂CH₂]ₙ—OR⁴. For such prodrugs, R⁴ is —H.

According to another embodiment, the cannabinoid prodrug is a compound in which, R¹ is —COOH and R² is propyl and —Z is -hemisuccinate.

Variable “Y” is any group selected from L-amino acid residue, a D-amino acid residue, a β-amino acid residue, a γ-amino acid residue, —C(O)—CH₂—[OCH₂CH₂]ₙ—O—, and —C(O)—CH₂—[OCH₂CH₂]ₙ—NH—.

Accordingly, in one embodiment, —Y—Z is an L-amino acid-hemisuccinate group, or a D-amino acid-hemisuccinate group. For prodrugs according to this embodiment, exemplary —Y—Z combinations include Gly-hemisuccinate, Ala-hemisuccinate, Val-hemisuccinate, Lys-hemisuccinate, D-Gly-hemisuccinate, D-Ala-hemisuccinate, D-Val-hemisuccinate, and D-Lys-hemisuccinate.

When variable “Y” is an L-amino acid, suitable examples include without limitation the twenty naturally occurring L-amino acids. When variable “Y” is a D-amino acid, exemplary D-amino acids include D-glycine, D-alanine D-valine, D-isoleucine, D-leucine, D-methionine, D-phenylalanine, D-tyrosine, D-tryptophan, D-serine, D-threonine, D-asparagine, D-glutamine, D-cysteine, D-arginine, D-histidine, D-lysine, D-aspartic acid, D-glutamic acid and D-proline.

In one embodiment, —Y is a β-amino acid and —Z is a -hemisuccinate. For such prodrugs, exemplary β-amino acids include without limitation β-phenylalanine, β-alanine, 3-aminobutanoic acid, 3-amino-3(3-bromophenyl)propionic acid, 2-amino-3-cyclopentene-1-carboxylic acid, 3-aminoisobutyric acid, 3-amino-2-phenylpropionic acid, 4,4-biphenylbutyric acid, 3-aminocyclohexanecarboxylic acid, 3-aminocyclopentanecarboxylic acid, and 2-aminoethylphenylacetic acid.

For some prodrugs, —Y is a γ-amino acid and —Z is -hemisuccinate. Illustrative γ-amino acids include γ-aminobutyric acid, statine, 4-amino-3-hydroxybutanoic acid, and 4-amino-3-phenylbutanoic acid (baclofen).

In one embodiment, —Z is an -oligosaccharide and illustrative “Y” groups include without limitation —C(O)—CH₂—[OCH₂CH₂]ₙ—O—, a polyethylene glycol moiety. The sugar moiety of an oligosaccharide prodrug can be a 5-member furanose, a 6-member pyroanose, a sugar with one or more of its hydroxyl groups protected by groups known in the chemical art. Alternatively, hydroxyl groups of the sugar moiety are unprotected. Both naturally occurring sugars and non-natural sugars that are chemically functionalized are used for the synthesis of cannabinoid prodrugs. Illustrate of the category of oligosaccharide prodrugs are mannose, N-acetyl glucosamine (GlcNAc), galactose, and sialic acid.

In one embodiment, the inventive prodrugs are de-carboxylated prior to their use as pharmaceutical or nutraceutical agents. Decarboxylation is achieved prior to contacting the Formula I or Formula II compound to an activated —Y—Z reagent under conditions suitable to effect the coupling of —Y—Z to the Formula I or Formula II compound. Alternatively, de-carboxylation is performed after synthesis of the prodrug, that is, using a Formula Ia or Formula IIa compound.

For certain prodrugs according to the invention, —Z is selected from —C(O)[CH₂]ₙ—NR⁴R⁵, —C(O)O[CH₂]ₙ—NR⁴R⁵, —C(O)—NH—[CH₂]ₙ—NR⁴R⁵, —C(O)[CH₂]ₙ—N⁺(R⁴)(R⁵))(R⁶)X⁻, —C(O)O[CH₂]ₙ—N⁺(R⁴)(R⁵)(R⁶)X⁻, or —C(O)—NH—[CH₂]ₙ—N⁺(R⁴)(R⁵)))(R⁶)X.

In one embodiment, —Y is the amino acid valine and —Y—Z is Val-NH—C(O)[CH₂]ₙ—NR⁴R⁵, Val-NH—C(O)O[CH₂]ₙ—NR⁴R⁵, or Val-NH—C(O)—NH—[CH₂]ₙ—NR⁴R⁵.

According to another embodiment, —Y is the amino acid lysine and —Y—Z is Lys-NH—C(O)[CH₂]ₙ—NR⁴R⁵, Lys-NH—C(O)O[CH₂]ₙ—NR⁴R⁵, or Lys-NH—C(O)—NH—[CH₂]ₙ—NR⁴R⁵. According to yet another embodiment, —Y is glutamic acid and —Y—Z is Glu-NH—C(O)[CH₂]ₙ—NR⁴R⁵, Glu-NH—C(O)O[CH₂]ₙ—NR⁴R⁵, or Glu-NH—C(O)—NH—[CH₂]ₙ—NR⁴R⁵.

In one embodiment, —Z is —C(O)[CH₂]ₙ—N⁺(R⁴)(R⁵))(R⁶)X⁻, —C(O)O[CH₂]ₙ—N⁺(R⁴)(R⁵)(R⁶)X⁻, or —C(O)—NH—[CH₂]ₙ—N⁺(R⁴)(R⁵))(R⁶)X⁻. Illustrative prodrugs are compounds where —Y—Z is -Val-NH—C(O)[CH₂]ₙ—N⁺(R⁴)(R⁵)(R⁶)X⁻, -Val-NH—C(O)O[CH₂]ₙ—N⁺(R⁴)(R⁵)(R⁶)X⁻, or -Val-NH—C(O)—NH—[CH₂]ₙ—N⁺(R⁴)(R⁵))(R⁶)X⁻, wherein R⁴, R⁵, and R⁶ are independently being from the group consisting of —H, —OH, formyl, acetyl, pivaloyl, and (C₁-C₅)alkyl, and subscript “n” is an integer between 1 and 6, inclusive of both integers. In one embodiment, “n” is 1, or 2. According to another embodiment, “n” is 3 and “X” is a counter ion derived from a pharmaceutically acceptable acid.

According to another embodiment, the Formula I, or II compound is obtained by contacting a Formula III compound with a cannabinoid synthase.

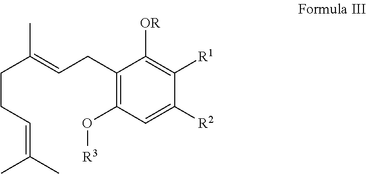

In one embodiment, the Formula I or Formula II compound is obtained when a Formula III compound is contacted with a cannabinoid synthase in the presence of a solvent. Solvents used for synthesis of prodrugs include without limitation aqueous buffer, a non-aqueous solvent, or a mixture comprising an aqueous buffer and a non-aqueous solvent. Buffers typically used in the method of the invention are citrate buffer, phosphate buffer, HEPES, Tris buffer, MOPS, or glycine buffer. Illustrative non-aqueous solvents include without limitation (C₁-C₅)alcohol, dimethyl sulfoxide (DMSO), dimethyl formamide (DMF), or iso-propoyl alcohol, β-cyclodextrin, and combinations thereof.

In one embodiment, the solvent is phosphate buffer, or citrate buffer. According to another embodiment, the solvent is TRIS buffer.

In one embodiment, the solvent is HEPES buffer, or a mixture of water and a (C₁-C₅)alcohol, or a mixture of buffer and a (C₁-C₅)alcohol. When the solvent is a mixture of an aqueous buffer and a non-aqueous solvent, the concentration of the non-aqueous solvent in the reaction mixture may vary between 10% and 50% (v/v), preferably the concentration of the non-aqueous solvent in the reaction mixture is 10%, 12%, 15%, 20%, 25%, 30%, 35%, 40%, 45%, or 50%. In one embodiment the concentration of the non-aqueous solvent in the reaction mixture is 30%. In another embodiment, the concentration of the non-aqueous solvent in the reaction mixture is 20%, or may vary between 10% and 20%, between 10% and 30%, or between 10% and 40%.

Cannabinoid acid synthase enzymes used to synthesize a cannabinoid prodrug according to the inventive method include without limitation tetrahydrocannabinolic acid synthase (THCA synthase), tetrahydrocannabivarin acid synthase (THCVA synthase), cannabidiolic acid synthase (CBDA synthase), or carmabichromene acid synthase (CBCA synthase). These enzymes may be obtained from natural sources or may be obtained by using any suitable recombinant method, including the use of the PichiaPink™ Yeast Expression system described in U.S. Provisional Application No. 62/041,521, filed Aug. 25, 2014 and U.S. patent application Ser. No. 14/835,444, filed Aug. 25, 2015 which published as U.S. Publication No.: 2016-0053220 on Feb. 26, 2016, the contents of which applications are incorporated by reference in their entireties.

Also encompassed by the disclosure is a method for producing a cannabinoid prodrug using a Formula VI compound as the substrate of a cannabinoid synthase.

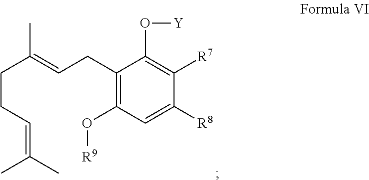

According to this method, contacting the Formula VI compound with a cannabinoid synthase produces a Formula IV or a Formula V compound:

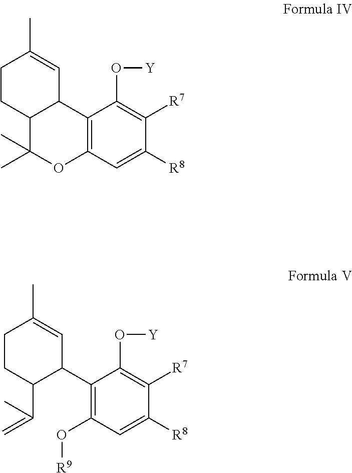

Further contact of the Formula IV or the Formula V compound with an activated reagent —Z (or activated —Z reagent) under conditions appropriate for coupling —Z to —Y provides Formula IVa or Formula Va prodrugs.

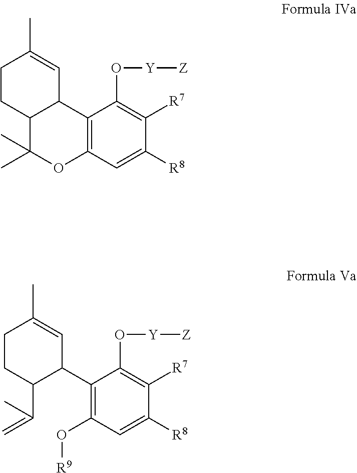

For Formula IV, IVa, V, Va, and VI compounds, R⁷ is —H, —COOH, or —COO(C₁-C₅)alkyl, and R⁸ is selected from the group consisting of (C₁-C₁₀)alkyl, (C₂-C₁₀)alkenyl, (C₂-C₁₀)alkynyl, (C₃-C₁₀)cycloalkyl, (C₃-C₁₀)cycloalkylalkylene, (C₃-C₁₀)aryl, and (C₃-C₁₀)arylalkylene.

Substituent R⁹ in Formula V, Va, and VI is —H, or (C₁-C₅)alkyl. Variable —Y in Formula IV, V, or VI is a group selected from L-amino acid residue, a D-amino acid residue, a β-amino acid residue, a γ-amino acid residue, and —C(O)—CH₂—[OCH₂CH₂]ₙ—NH—, while variable —Z is selected from hemisuccinate, succinate, oxalate, —C(O)—CH₂—[OCH₂CH₂]ₙ—OR¹⁰, —C(O)—CH₂—[OCH₂CH₂]ₙ—NH₂, —C(O)[CH₂]ₙ—NR¹⁰R¹¹, —C(O)O[CH₂]ₙ—NR¹⁰R¹¹, —C(O)—NH—[CH₂]ₙ—NR¹⁰R¹¹, —C(O)[CH₂]ₙ—N⁺(R¹⁰)(R¹¹))(R¹²)X⁻, —C(O)O[CH₂]ₙ—N⁺(R¹⁰)(R¹¹)(R¹²)X⁻, and —C(O)—NH—[CH₂]ₙ—N⁺(R¹⁰)(R¹¹))(R¹²)X⁻.

For Formula IVa and Va compounds, —Y—Z includes without limitation —Y-hemisuccinate, —Y-succinate, —Y-oxalate, —Y—C(O)—CH₂—[OCH₂CH₂]ₙ—OR¹⁰, —Y—C(O)—CH₂—[OCH₂CH₂]ₙ—NH₂, —Y—C(O)[CH₂]ₙ—NR¹⁰R¹¹, —Y—C(O)O[CH₂]ₙ—NR¹⁰R¹¹, —Y—C(O)—NH—[CH₂]ₙ—NR¹⁰R¹¹, —Y—C(O)[CH₂]ₙ—N⁺(R¹⁰)(R¹¹))(R¹²)X⁻, —Y—C(O)O[CH₂]ₙ—N⁺(R¹⁰)(R¹¹)(R¹²)X⁻, and —Y—C(O)—NH—[CH₂]ₙ—N⁺(R¹⁰)(R¹¹)(R¹²)X⁻.

In one embodiment, R⁷ is —COOH and R⁸ is a (C₁-C₅)alkyl, such as propyl or pentyl, —Y is an amino acid and —Z is -hemisuccinate. Exemplary compounds are those wherein —Y—Z is selected from Val-hemisuccinate, Lys-hemisuccinate, Ala-hemisuccinate, Glu-hemisuccinate, Pro-hemisuccinate, and Asp-hemisuccinate.

For some cannabinoid prodrugs of the invention, —Z is -succinate, —C(O)—CH₂—[OCH₂CH₂]ₙ—OR¹⁰, or —C(O)—CH₂—[OCH₂CH₂]ₙ—NH₂.

In one embodiment, R⁷ is —COOH and R⁸ is propyl or pentyl and —Z is —C(O)—CH₂—[OCH₂CH₂]ₙ—OR¹⁰. For such prodrugs, R¹⁰ is —H, methyl, ethyl, propyl, iso-propyl or t-butyl.

For certain other prodrugs, —Y is valine and —Z is a —C(O)—CH₂—[OCH₂CH₂]ₙ—NH₂ or a —C(O)—NH—[CH₂]ₙ—N⁺(R¹⁰)(R¹¹))(R¹²)X⁻ group. Illustrative groups include Val-NH—C(O)—CH₂—[OCH₂CH₂]ₙ—NH₂ and -Val-NH—C(O)—NH—[CH₂]ₙ—N⁺(R¹⁰)(R¹¹)(R¹²)X⁻ group.

In one embodiment, —Z is —C(O)[CH₂]ₙ—NR¹⁰R¹¹, —C(O)O[CH₂]ₙ—NR¹⁰R¹¹, —C(O)—NH—[CH₂]ₙ—NR¹⁰R¹¹, —C(O)[CH₂]ₙ—N⁺(R¹⁰)(R¹¹))(R¹²)X⁻, —C(O)O[CH₂]ₙ—N⁺(R¹⁰)(R¹¹)(R¹²)X⁻, “X” is a counter ion derived from a pharmaceutically acceptable acid, and substituents R¹⁰, R¹¹, and R¹² are each independently selected from the group consisting of —H, —OH, formyl, acetyl, pivaloyl, and (C₁-C₅)alkyl.

In one embodiment, the compound according to Formula IV or Formula V is directly used as a cannabinoid prodrug and can be formulated in a suitable pharmaceutically acceptable formulation.

During the manufacture of a prodrug according to this method, the step of contacting a Formula VI compound with a cannabinoid synthase can take place in the presence of a solvent. Illustrative solvents without limitation include aqueous and organic solvent solvents as well as mixtures thereof. In one embodiment, the solvent is water, cylodextrin, phosphate buffer, dimethyl sulfoxide (DMSO), citrate buffer, TRIS buffer, HEPES buffer, a mixture of water and a (C₁-C₅)alcohol, and a mixture of buffer and a (C₁-C₅)alcohol.

The inventors of the present application have unexpectedly discovered that ⁻the concentration of the non-aqueous solvent in the reaction mixture affects the rate of the enzyme-catalyzed reaction as well as the ratio of the cannabinoid prodrug obtained as products. For example, it was observed that the presence of cyclodextrins, cyclic oligosaccharides that are amphiphilic and function as surfactants, accelerates the rate of the enzyme catalyzed cyclization reaction of a Formula III, VI or IX compounds (substrates) to the corresponding cannabinoid compounds or cannabinoid prodrugs (products). It was surprising to note that the concentration of cyclodextrin in the reaction mixture also affects product ratio, that is, the ratio of the amount of a Formula II compound to the amount of a Formula III compound produced using the inventive method.

Another surprising and unexpected observation was that pH of the reaction mixture affects the ratio of the cannabinoid prodrugs produced using the inventive method. In one preferred embodiment, a Formula VI and Formula IX compounds according to the invention are contacted with THCA synthase produces a prodrug of a tetrahydrocannabinolic acid (THCA) or a prodrug of a cannabichromene acid (CBCA), in different ratios depending on the pH of the reaction mixture.

Thus in one its embodiments the invention provides a method for producing cannabinoid prodrugs at varying pH values of the reaction mixture. In one example, the bioenzymatic synthesis of a prodrug is performed at a pH in a range between 3.0 and 8.0, for example at a pH in a range between 3.0 and 7.0, between 3.0 and 6.0, between 3.0 and 5.0, or between 3.0 and 4.0.

In one embodiment, the reaction is performed at a pH in a range between 3.8 and 7.2. According to another embodiment, the reaction is performed at a pH in a range between 3.5 and 8.0, between 3.5 and 7.5, between 3.5 and 7.0, between 3.5 and 6.5, between 3.5 and 6.0, between 3.5 and 5.5, between 3.5 and 5.0, or between 3.5 and 4.5.

Exemplary pharmaceutically acceptable acids (X⁻), include without limitation formic, acetic, propionic, succinic, glycolic, gluconic, lactic, malic, tartaric, citric, ascorbic, glucuronic, maleic, fumaric, pyruvic, aspartic, glutamic, benzoic, anthranilic, mesylic, stearic, salicylic, p-hydroxybenzoic, phenylacetic, mandelic, embonic, methanesulfonic, ethanesulfonic, benzenesulfonic, pantothenic, toluenesulfonic, 2-hydroxyethanesulfonic, sulfanilic, cyclohexylaminosulfonic, algenic, beta-hydroxybutyric, galactaric and galacturonic acids. The list of pharmaceutically acceptable salts mentioned above is not meant to be exhaustive but merely illustrative, because a person of ordinary skill in the art would appreciate that other pharmaceutically acceptable salts of a prodrug of a cannabinoid and can be prepared using methods known in the formulary arts.

For example, acid addition salts are readily prepared from a free base by reacting the free base with a suitable acid. Suitable acids for preparing acid addition salts include both (i) organic acids, for example, formic acid, acetic acid, propionic acid, glycolic acid, pyruvic acid, oxalic acid, malic acid, malonic acid, succinic acid, maleic acid, fumaric acid, tartaric acid, citric acid, benzoic acid, cinnamic acid, mandelic acid, methanesulfonic acid, ethanesulfonic acid, p-toluenesulfonic acid, salicylic acid, and the like, and (ii) inorganic acids, for example, hydrochloric acid, hydrobromic acid, sulfuric acid, nitric acid, phosphoric acid, and the like.

The present also provides a method for producing a cannabinoid prodrug by the enzyme-catalyzed conversion of a substrate that is modified to comprise the prodrug moiety. Accordingly, in one embodiment the inventive method provides prodrugs according to Formula VIIa or Formula VIIIa.

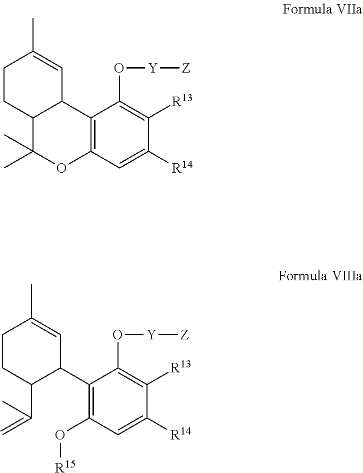

According to this embodiment of the inventive method, Formula VIIa and VIIIa prodrugs are obtained by contacting a compound of Formula IX with a cannabinoid synthase.

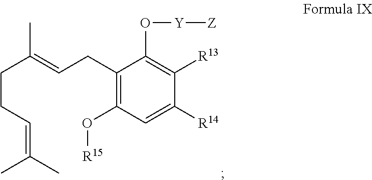

For Formula VIIa, VIIIa and IX compounds, substituent R¹³ is —H, —COOH, or —COO(C₁-C₅)alkyl, substituent R¹⁴ is selected from the group consisting of (C₁-C₁₀)alkyl, (C₂-C₁₀)alkenyl, (C₂-C₁₀)alkynyl, (C₃-C₁₀)cycloalkyl, (C₃-C₁₀)cycloalkylalkylene, (C₃-C₁₀)aryl, and (C₃-C₁₀)arylalkylene, and R¹⁵ is either —H, or (C₁-C₅)alkyl.

For Formula VIIa, VIIIa and IX compounds, variable —Y is selected from L-amino acid residue, a D-amino acid residue, a β-amino acid residue, a γ-amino acid residue, —C(O)—CH₂—[OCH₂CH₂]ₙ—O— and —C(O)—CH₂—[OCH₂CH₂]ₙ—NH—.

Variable —Z in Formula VIIa, VIIIa and IX is a group selected from hemisuccinate, succinate, oxalate, —C(O)—CH₂—[OCH₂CH₂]ₙ—OR⁴, —C(O)—CH₂—[OCH₂CH₂]ₙ—NH₂, —C(O)[CH₂]ₙ—NR¹⁶R¹⁷, —C(O)O[CH₂]ₙ—NR¹⁶R¹⁷, —C(O)—NH—[CH₂]ₙ—NR¹⁶R¹⁷, —C(O)[CH₂]ₙ—N⁺(R¹⁶)(R¹⁷))(R¹⁸)X⁻, —C(O)O[CH₂]ₙ—N⁺(R¹⁶)(R¹⁷)(R¹⁸)X⁻, and —C(O)—NH—[CH₂]ₙ—N⁺(R¹⁶)(R¹⁷)(R¹⁸)X⁻ and subscript “n” is an integer, for example 1, 2, 3, 4, 5, or 6.

“X” is a counter ion derived from a pharmaceutically acceptable acid, while substituents R¹⁶, R¹⁷, and R¹⁸ are each independently selected from the group consisting of —H, —OH, formyl, acetyl, pivaloyl, and (C₁-C₅)alkyl.

In one embodiment, the cannabinoid synthase enzyme used to produce a prodrug according to Formula VIIa, or Formula VIIIa is tetrahydrocannabivarin acid synthase (THCVA synthase), tetrahydrocannabinolic acid synthase (THCA synthase), cannabidiolic acid synthase (CBDA synthase), or cannabichromene acid synthase (CBCA synthase).

In one embodiment, the enzyme is THCA synthase and the enzyme-catalyzed production of a Formula VIIa, or a Formula VIIIa compound is carried out at a pH from about 4.0 to about 8.0.

In one embodiment, the pH for the enzyme-catalyzed production of a Formula VIIa, or a Formula VIIIa compound is about 4.5, about 5.0, about 5.5, about 6.0, about 6.5. or about 7.0.

According to one embodiment, the pH for the enzyme-catalyzed production of a Formula VIIIa, or a Formula VIIIa compound is about 5.0.

According to yet another embodiment, the pH for the enzyme-catalyzed production of a Formula VIIa, or a Formula VIIIa compound is about 7.0.

For certain Formula VIIa and Formula VIIIa prodrugs, —Y—Z is an L-amino acid-hemisuccinate. For example, —Y—Z is Ala-hemisuccinate, Lys-hemisuccinate, Glu-hemisuccinate, Phe-hemisuccinate, Asp-hemisuccinate, or Gly-hemisuccinate. For certain other Formula VIIa and Formula VIIIa prodrugs, —Y—Z is a D-amino acid-hemisuccinate.

The prodrug of a cannabinoid or a cannabinoid analog synthesized according to a method of the invention may be purified prior to use. Purification is effected by procedures routinely used in the chemical and biochemical art, including solvent extraction or chromatographic purification methods. The purity of the purified prodrug product can be determined by thin layer chromatography (TLC), High Performance Liquid Chromatography coupled to a mass spectrometer (HPLC-MS), or by any suitable analytical technique. Nuclear magnetic resonance spectroscopy, mass spectral analysis, or UV, visible spectroscopy, are examples of analytical methods that can be used to confirm the identity of the inventive prodrugs.

Typically, the enantiomeric purity of the inventive prodrugs is from about 90% ee to about 100% ee, for instance, a prodrug of a cannabinoid or a cannabinoid analog according to the present invention can have an enantiomeric purity of about 91% ee, about 92% ee, about 93% ee, about 94% ee, about 95% ee, about 96% ee, about 97% ee, about 98% ee and about 99% ee. Cannabinoids exert different physiological properties and are known to lessen pain, stimulate appetite and have been tested as candidate therapeutics for treating a variety of disease conditions such as allergies, inflammation, infection, epilepsy, depression, migraine, bipolar disorders, anxiety disorder, and glaucoma. The physiological effects exerted by cannabinoids is affected by their ability to stimulate or deactivate the cannabinoid receptors, for instance the CB1, CB2 and CB3 receptors.

### Pharmaceutical Compositions

The prodrugs synthesized using the inventive methods are administered to a patient or subject in need of treatment either alone or in combination with other compounds having similar or different biological activities. For example, the prodrugs and composition comprising the prodrugs of the invention can be administered in a combination therapy, i.e., either simultaneously in single or separate dosage forms or in separate dosage forms within hours or days of each other. Examples of such combination therapies include administering a composition comprising a prodrug according Formula Ia, IIa, IV, V, IVa, Va, VIIa, or VIIIa with other pharmaceutical agents used to treat glaucoma, AIDS wasting, neuropathic pain, treatment of spasticity associated with multiple sclerosis, fibromyalgia and chemotherapy-induced nausea, emesis, wasting syndrome, HIV-wasting, alcohol use disorders, dystonia, multiple sclerosis, inflammatory bowel disorders, arthritis, dermatitis, Rheumatoid arthritis, systemic lupus erythematosus, anti-inflammatory, anti-convulsant, anti-psychotic, antioxidant, neuroprotective, anti-cancer, immunomodulatory effects, peripheral neuropathic pain, neuropathic pain associated with post-herpetic neuralgia, diabetic neuropathy, shingles, burns, actinic keratosis, oral cavity sores and ulcers, post-episiotomy pain, psoriasis, pruritic, contact dermatitis, eczema, bullous dermatitis herpetiformis, exfoliative dermatitis, mycosis fungoides, pemphigus, severe erythema multiforme (e.g., Stevens-Johnson syndrome), seborrheic dermatitis, ankylosing spondylitis, psoriatic arthritis, Reiter's syndrome, gout, chondrocalcinosis, joint pain secondary to dysmenorrhea, fibromyalgia, musculoskeletal pain, neuropathic-postoperative complications, polymyositis, acute nonspecific tenosynovitis, bursitis, epicondylitis, post-traumatic osteoarthritis, osteoarthritis, rheumatoid arthritis, synovitis, juvenile rheumatoid arthritis and inhibition of hair growth.

The invention also provides a pharmaceutical composition comprising a pharmaceutically acceptable salt, solvate, or stereoisomer of a prodrug according to invention in admixture with a pharmaceutically acceptable carrier. In some embodiments, the composition further contains, in accordance with accepted practices of pharmaceutical compounding, one or more additional therapeutic agents, pharmaceutically acceptable excipients, diluents, adjuvants, stabilizers, emulsifiers, preservatives, colorants, buffers, flavor imparting agents.

The inventive compositions can be administered orally, topically, parenterally, by inhalation or spray or rectally in dosage unit formulations. The term parenteral as used herein includes subcutaneous injections, intravenous, intramuscular, intrastemal injection or infusion techniques.

Suitable oral compositions in accordance with the invention include without limitation tablets, troches, lozenges, aqueous or oily suspensions, dispersible powders or granules, emulsion, hard or soft capsules, syrups or elixirs.

Encompassed within the scope of the invention are pharmaceutical compositions suitable for single unit dosages that comprise a prodrug of the invention its pharmaceutically acceptable stereoisomer, salt, solvate, hydrate, or tautomer and a pharmaceutically acceptable carrier.

Inventive compositions suitable for oral use may be prepared according to any method known to the art for the manufacture of pharmaceutical compositions. For instance, liquid formulations of the inventive prodrugs contain one or more agents selected from the group consisting of sweetening agents, flavoring agents, coloring agents and preserving agents in order to provide pharmaceutically elegant and palatable preparations of the inventive prodrug.

For tablet compositions, the active ingredient in admixture with non-toxic pharmaceutically acceptable excipients is used for the manufacture of tablets. Exemplary of such excipients include without limitation inert diluents, such as calcium carbonate, sodium carbonate, lactose, calcium phosphate or sodium phosphate; granulating and disintegrating agents, for example, corn starch, or alginic acid; binding agents, for example starch, gelatin or acacia, and lubricating agents, for example magnesium stearate, stearic acid or talc. The tablets may be uncoated or they may be coated by known coating techniques to delay disintegration and absorption in the gastrointestinal tract and thereby to provide a sustained therapeutic action over a desired time period. For example, a time delay material such as glyceryl monostearate or glyceryl di-stearate may be employed.

Formulations for oral use may also be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example peanut oil, liquid paraffin or olive oil.

For aqueous suspensions, the inventive prodrug is admixed with excipients suitable for maintaining a stable suspension. Examples of such excipients include without limitation are sodium carboxymethylcellulose, methylcellulose, hydropropylmethylcellulose, sodium alginate, polyvinylpyrrolidone, gum tragacanth and gum acacia.

Oral suspensions can also contain dispersing or wetting agents, such as naturally occurring phosphatide, for example, lecithin, polyoxyethylene stearate, heptadecaethyleneoxycetanol, polyoxyethylene sorbitol monooleate, polyethylene sorbitan monooleate. The aqueous suspensions may also contain one or more preservatives, for example ethyl, or n-propyl p-hydroxybenzoate, one or more coloring agents, one or more flavoring agents, and one or more sweetening agents, such as sucrose or saccharin.

Oily suspensions may be formulated by suspending the prodrug in a vegetable oil, for example arachis oil, olive oil, sesame oil or coconut oil, or in a mineral oil such as liquid paraffin. The oily suspensions may contain a thickening agent, for example beeswax, hard paraffin or cetyl alcohol.

Syrups and elixirs may be formulated with sweetening agents, for example glycerol, propylene glycol, sorbitol or sucrose. Such formulations may also contain a demulcent, a preservative, and flavoring and coloring agents. The pharmaceutical compositions may be in the form of a sterile injectable, or an aqueous suspension. This suspension may be formulated according to the known art using those suitable dispersing or wetting agents and suspending agents. The sterile injectable preparation may also be sterile injectable solution or suspension in a non-toxic parentally acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium. For this purpose any bland fixed oil may be employed including synthetic mono- or diglycerides. In addition, fatty acids such as oleic acid find use in the preparation of injectables.

Compositions for parenteral administrations are administered in a sterile medium. Depending on the vehicle used and concentration the concentration of the drug in the formulation, the parenteral formulation can either be a suspension or a solution containing dissolved drug. Adjuvants such as local anesthetics, preservatives and buffering agents can also be added to parenteral compositions.

The total amount by weight of a cannabinoid prodrug of the invention in a pharmaceutical composition is from about 0.1% to about 95%. By way of illustration, the amount of a cannabinoid prodrug by weight of the pharmaceutical composition, such as a cannabidiol prodrug, a THC prodrug, or a THC-v prodrug of the invention can be about 0.1%, about 0.2%, about 0.3%, about 0.4%, about 0.5%, about 0.6%, about 0.7%, about 0.8%, about 0.9%, about 1%, about 1.1%, about 1.2%, about 1.3%, about 1.4%, about 1.5%, about 1.6%, about 1.7%, about 1.8%, about 1.9%, about 2%, about 2.1%, about 2.2%, about 2.3%, about 2.4%, about 2.5%, about 2.6%, about 2.7%, about 2.8%, about 2.9%, about 3%, about 3.1%, about 3.2%, about 3.3%, about 3.4%, about 3.5%, about 3.6%, about 3.7%, about 3.8%, about 3.9%, about 4%, about 4.1%, about 4.2%, about 4.3%, about 4.4%, about 4.5%, about 4.6%, about 4.7%, about 4.8%, about 4.9%, about 5%, about 5.1%, about 5.2%, about 5.3%, about 5.4%, about 5.5%, about 5.6%, about 5.7%, about 5.8%, about 5.9%, about 6%, about 6.1%, about 6.2%, about 6.3%, about 6.4%, about 6.5%, about 6.6%, about 6.7%, about 6.8%, about 6.9%, about 7%, about 7.1%, about 7.2%, about 7.3%, about 7.4%, about 7.5%, about 7.6%, about 7.7%, about 7.8%, about 7.9%, about 8%, about 8.1%, about 8.2%, about 8.3%, about 8.4%, about 8.5%, about 8.6%, about 8.7%, about 8.8%, about 8.9%, about 9%, about 9.1%, about 9.2%, about 9.3%, about 9.4%, about 9.5%, about 9.6%, about 9.7%, about 9.8%, about 9.9%, about 10%, about 11%, about 12%, about 13%, about 14%, about 15%, about 16%, about 17%, about 18%, about 19%, about 20%, about 25%, about 30%, about 35%, about 40%, about 45%, about 50%, about 55%, about 60%, about 65%, about 70%, about 75%, about 80%, about 85%, about 90% or about 95%.

In one embodiment, the pharmaceutical composition comprises a total amount by weight of a cannabinoid prodrug, of about 1% to about 10%; about 2% to about 10%; about 3% to about 10%; about 4% to about 10%; about 5% to about 10%; about 6% to about 10%; about 7% to about 10%; about 8% to about 10%; about 9% to about 10%; about 1% to about 9%; about 2% to about 9%; about 3% to about 9%; about 4% to about 9%; about 5% to about 9%; about 6% to about 9%; about 7% to about 9%; about 8% to about 9%; about 1% to about 8%; about 2% to about 8%; about 3% to about 8%; about 4% to about 8%; about 5% to about 8%; about 6% to about 8%; about 7% to about 8%; about 1% to about 7%; about 2% to about 7%; about 3% to about 7%; about 4% to about 7%; about 5% to about 7%; about 6% to about 7%; about 1% to about 6%; about 2% to about 6%; about 3% to about 6%; about 4% to about 6%; about 5% to about 6%; about 1% to about 5%; about 2% to about 5%; about 3% to about 5%; about 4% to about 5%; about 1% to about 4%; about 2% to about 4%; about 3% to about 4%; about 1% to about 3%; about 2% to about 3%; or about 1% to about 2%.

### EXAMPLES

### A. Chemical Synthesis

### I. Synthesis of Olivetol

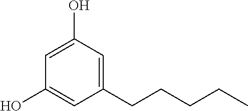

Olivetol was synthesized using a published procedure (Focella, A, et al., J. Org. Chem., Vol. 42, No. 21, (1977), p. 3456-3457).

### i. Methyl 6-N-Pentyl-2-hydroxy-4-oxo-cyclohex-2-ene-l-carboxylate

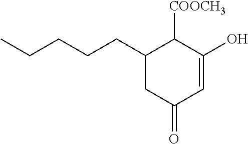 [SMILES: CCCCCC1CC(=O)C=C(O)C1C(=O)OC]

To a stirring solution of sodium methoxide (32.4 g, 0.60 mol) and dimethyl malonate (90 g, 0.68 mol) in 230 mL of anhydrous methanol was added portion wise 75 g (0.48 mol) of 90% 3-nonen-2-one. The reaction mixture was then refluxed for 3 h under N₂ and allowed to cool to room temperature. The solvent was distilled under reduced pressure and the residue dissolved in 350 mL of water. The slurry of white crystals and the almost clear solution was extracted thrice with 80 mL of chloroform. The aqueous layer was acidified to pH 4 with concentrated HCl and the white precipitate that formed was allowed to stand overnight prior to filtration. The crystals were dried at 50° C. under high vacuum for 5 hours to yield 106.5 g (0.4416 mol) (92%) of methyl 6-n-Pentyl-2-hydroxy-4-oxo-cyclohex-2-ene-l-carboxylate (mp 96-98 C). The product was recrystallized using a mixture of petroleum ether:ethyl acetate (9:1), and gave 94 g of pure methyl 6-n-Pentyl-2-hydroxy-4-oxo-cyclohex-2-ene-l-carboxylate (melting point of 98-100 C).

### ii. 1-n-Penyl-3,5-dihydroxybenzene (Olivetol)

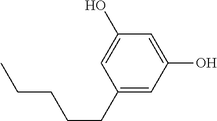

To a stirring ice-cooled solution of methyl 6-N-pentyl-2-hydroxy-4-oxo-cyclohex-2-ene-l-carboxylate (58.4 g, 0.24 mol) dissolved in 115 mL dimethylformamide was added dropwise 37.9 g (0.23 mol) of bromine dissolved in 60 mL of dimethylformamide. At the end of the addition (ca. 90 min) the reaction mixture was slowly heated to 80° C. during which time the evolution of carbon dioxide became quite vigorous.

The reaction was maintained at this temperature until gas evolution had ceased following which the reaction was further heated to 160° C. and held at this temperature for approximately 10 hours. After heating, the reaction was allowed to cool and the solvent DMF was removed under reduced pressure. The residue thus obtained was treated with water (80 mL) and extracted twice with 250 mL of ether. The combined ether layers were washed with water, then washed with 2×80 mL of a 10% solution of sodium bisultite, 2×80 mL of a 10% solution of acetic acid, and then again with water.

After drying over anhydrous sodium sulfate the solvent was removed under reduced pressure to give 46.8 g of viscous oil. The oil was distilled under reduced pressure to give 30.3 g (0.168 mol) (69.3%) of olivetol as product. HPLC analysis indicated 97.5% purity.

### II. Synthesis of CBG

CBG was synthesized following the protocol disclosed by Taura et al., (1996), The Journal of Biological Chemistry, Vol. 271, No. 21, p. 17411-17416.

### Synthesis of 2-[(2E)-3,7-dimethylocta-2,6-dienyl]-5-pentyl-benzene-1,3-diol (Cannabigerol (CBG))

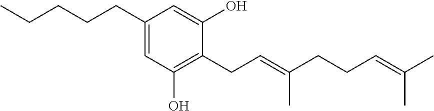 [SMILES: CCCCCc1cc(O)c(C/C=C(\C)CCC=C(C)C)c(O)c1]

Geraniol (3 g, 0.0194 mol) and olivetol (2 g, 0.0111 mol) were dissolved in 400 mL of chloroform containing 80 mg of p-toluenesulfonic acid as catalyst and the reaction mixture was stirred at room temperature for 12 h in the dark. After 12 hours, the reaction mixture was washed with saturated sodium bicarbonate (400 mL) and then with H₂O (400 mL). The chloroform layer was concentrated at 40° C. under reduced pressure, and the residue obtained was chromatographed on a 2.0 cm×25 cm silica gel column using benzene (1000 mL) as the eluent to give 1.4 g (0.00442 mol) (39.9%) CBG as product.

Alternatively crude CBG was purified as follows. To a 250 mL beaker was added 7.25 g crude CBG and 50 mL benzene. The flask was swirled to dissolve the CBG and 50 g silica gel was added, along with a stir bar. The solution was stirred overnight, and then poured into a 44 cm×2.75 cm column. The column was eluted with 300 mL benzene. The eluent, approximately 70 mL fractions were assayed for CBG. Fractions 1, 2, and 3 (˜230 mL) that contained CBG were combined and the solvent removed under pressure to give 6.464 g residue containing >80% CBG, having a purity suitable for use in the next synthetic step.

In one embodiment, crude CBG was purified by mixing 7.25 g crude CBG residue with a slurry of silica gel (50 mL), in a 250 ml Beaker. This mixture was slowly agitated for 1 hour and then vacuum filtered using a fine mesh filter paper. The filter cake was washed with 250 ml benzene until a clear filtrate was obtained. The solvent from the filtrate was removed under reduced pressure to give 6.567 g of a residue having >80% CBG.

### III. Synthesis of Methylmagnesium Carbonate (MMC)

Methylmagnesium Carbonate (MMC) was synthesized following the protocol disclosed by Balasubrahmanyam et al., (1973), Organic Synthesis, Collective Volume V, John Wiley & Sons, Inc., p. 439-444.

A dry 2 L, three necked flask was fitted with a mechanical stirrer, a condenser, and a 1 L, pressure-equalizing addition funnel, the top of which was fitted with a gas inlet tube. A clean, dry magnesium ribbon (40.0 g, 1.65 mol) was placed in the flask and the system was flushed with nitrogen prior to the addition of anhydrous methanol (600 mL). The evolution of hydrogen gas was controlled by cooling the reaction mixture. When evolution of hydrogen gas ceased, a slow stream of nitrogen was passed through the system and the condenser replaced by a total condensation-partial take-off distillation head. The nitrogen flow was stopped and the bulk of the methanol distilled from the solution under reduced pressure. Distillation was stopped when stirring of the pasty suspension of magnesium methoxide was no longer practical. The system was again flushed using nitrogen and the outlet from the distillation head was attached to a small trap containing mineral oil so that the volume of gas escaping from the reaction system could be estimated.

Anhydrous dimethylformamide (DMF) (700 mL) was added to the reaction flask, and the resulting suspension was stirred vigorously while a stream of anhydrous carbon dioxide was passed into the reaction vessel through the gas inlet tube attached to the addition funnel. The dissolution of carbon dioxide was accompanied by an exothertnic reaction with the suspended magnesium methoxide. When no more CO₂ is absorbed, the colorless solution was heated under a slow stream of CO₂ gas until the temperature of the liquid distilling reached 140° C., indicating that residual methanol had been removed from the reaction mixture. The reaction mixture was flushed using a slow stream of nitrogen to aid in cooling the mixture to room temperature under an inert atmosphere. This yielded a solution aving 536 mg MMC/mL of DMF.

### IV. Synthesis of CBGA (3-[3,7-dimethyl-2,6-octadiene]-2,4-dihydroxy-6-pentyl benzene-1-carboxylic acid)

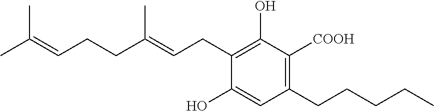 [SMILES: CCCCCc1cc(O)c(C/C=C(\C)CCC=C(C)C)c(O)c1C(=O)O]

6-carboxylic acid-2-[(2E)-3,7-dimethylocta-2,6-dienyl]-5-pentyl-benzene-1,3-diol, Cannabigerolic Acid (CBGA) was prepared as follows. To a 10 mL conical flask was added 1 mL of a DMF solution of MMC. To this solution was added 2-[(2E)-3,7-dimethylocta-2,6-dienyl]-5-pentyl-benzene-1,3-diol (120 mg, 0.379 mmol). The flask was heated at 120° C. for 1 hour, following which the reaction mixture was dissolved in 100 mL of chloroform:methanol (2:1) solution. The pH of this solution was adjusted with dilute HCl to pH 2.0, and then partitioned using 50 mL H₂O.

The organic layer was dried over sodium sulfate and the solvent was removed by evaporation. HPLC analysis of the crude reaction showed ˜40% conversion of CBG to CBGA.

Alternatively, 3.16 g (10 mmols) of CBG (or any other neutral cannabinoid), 8.63 g (100 mmols) magnesium methylate and 44 g (1 mol) of dry ice were sealed in a pressure compatible vessel. The vessel is heated to 50° C., and the temperature held at this value for three hours. Following heating, the vessel is cooled to room temperature and slowly vented. The reaction mixture was dissolved in 100 mL of a chloroform: methanol (2:1) solvent. The pH of this solution was adjusted with dilute HCl to pH 2.0 and this solution was then partitioned using 50 mL of H₂O. The organic layer was dried over sodium sulfate and the solvent was removed by evaporation. HPLC analysis of crude reaction mixture showed ˜85% conversion of CBG to CBGA using this protocol.

Crude CBGA was purified by chromatography using a 2.0 cm×25 cm silica gel column. The product was eluted using a mixture of n-hexane:ethyl acetate (2:1) (1000 mL), to obtain 45 mg (0.125 mmol) (37.5%) of the desired product.

Alternatively, ultra high purity CBGA was obtained by chromatographing the crude using LH-20 lipophilic resin as the medium. 400 g of LH-20 Sephadex resin was first swollen using 2 L of DCM:chloroform (4:1) solvent. The swollen resin was gravity packed in a 44×2.75 cm column. The column was loaded with 2.1 g of crude CBGA dissolved in a minimum amount of DCM:chloroform (4:1) solvent and eluted with 1.7 L of the same solvent. 100 mL fractions were collected. The unreacted CBG was eluted as a yellow/orange solution using this solvent system. After the passage of about 1.7 L of this solvent, no more yellow/orange fraction were observed and the eluting solvent was changed to 100% acetone to elute the bound CBGA.

The fractions containing CBGA were pooled and the solvent was removed to obtain 0.52 g CBGA (˜90% recovery). Increasing the volume of DCM:chloroform (4:1) solvent passed through the column prior to eluting with acetone, yielded CBGA having purity greater than 99.5%.

### V. Synthesis of TBDMS-CBGA (3-[3,7-dimethylocta-2,6-diene]-2-hydroxy-6-pentyl -4-[t-butyldimethylsilyloxy]benzoic acid) or TBDMS-CBGA-methy/ethyl ester (Methyl-/Ethyl-3-[3,7-dimethylocta-2,6-diene]-2-hydroxy-6-pentyl-4-[t-butyldimethylsilyloxy]benzoate)

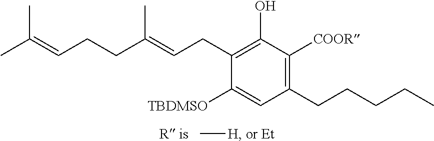

To a cold stirring solution of CBGA or CBGA-ethyl ester in DCM under an atmosphere of argon is added t-butyldimethylsilyl chloride (1.0 eq.) and imidazole. TLC is used to monitor reaction progress. The reaction is quenched upon completion by the addition of brine. The organic layer was separated and dried using anhydrous magnesium sulfate prior to purification and use. If CBGA-ethyl ester is used as the starting material, the product can be hydrolyzed to the corresponding acid, if necessary, prior to enzyme-catalyzed synthesis of the cannabinoid prodrug.

A similar protocol is used for synthesizing 3-[3,7-dimethylocta-2,6-diene]-2-hydroxy-6-pentyl-4-[trimethylsilyloxy]benzoic acid via the reaction of CBGA or CBGA-ester with trimethylsilyl chloride in the presence of a base such as imidazole.

### B. Synthesis of Activated —Y—Z Reagent

### Synthesis of THCA-Val-hemisuccinate Methyl/Allyl Ester

i. Synthesis of Resin-Val-NH(Fmoc)

Rink acid resin (0.3-1.5 umoles/g of resin) or 2-chlorotrilyl resin (2-Trt) resin (Advance ChemTech), is swollen in a solvent mixture of DCM/DMF, for 30 minutes. After swelling, the resin is washed with DMF (2×), following which, a DMF solution of (Fmoc)Val-OH (5× w.r.t resin loading capacity) is added to the resin. After stirring for about 5 minutes DIEA or 2,4,6-collidine (˜5-fold w.r.t. Fmoc-Val-OH) is added. A small amount of DCM or methanol is added as needed to solubilize the reactants. The resin-amino acid solution is stirred, or agitated by bubbling nitrogen gas for about 3 h. After stirring for about 3 h, a known amount of resin is withdrawn and placed into a small test tube. The resin is washed with DMF (3×), then DCM (3×) and finally with methanol (2×). The resin is dried using a gentle stream of nitrogen, and then cleaved using a 1% solution of TFA in DCM. The amino acid loading is determined by HPLC, by quantifying the amount of Fmoc-Val in the TFA-DCM solution used to cleave a known amount of the resin. If loading of the amino acid is incomplete, it is necessary to repeat the above-described protocol.

ii. De-Protection of Fmoc

The (Fmoc)Val-resin is swollen in DMF or NMP for about 30-45 minutes prior to removal of the Fmoc group. After draining the DMF solution, (Fmoc)Val-resin is contacted with a 20% solution of piperidine in NMP (or DMF). After stirring for about 30 min., a small amount of the resin is removed into a test tube. The resin is washed with DMF (2×) and checked for removal of the Fmoc group using the ninhydrin test for detection of free amines.

iii. Coupling of Succinic Acid

The deprotected NH₂-Val-resin is washed with DMF (×3), DCM (×2), and then the resin is re-suspended in DMF (or NMP). To the DMF-resin slurry is added a. DMF solution of succinic anhydride (5 eq. w.r.t. resin loading capacity), followed by the addition of DIEA (5 eq.). The mixture is stirred for about 60-90 min., and then a small amount of the resin is withdrawn into a clean test tube. The withdrawn resin is washed with DMF (2×), DCM (2×), and finally with methanol. Checking the coupling of succinic acid to NH₂-Val-resin is performed with the ninhydrin test. A negative test indicates complete coupling. However, if coupling of succinic acid to NH₂-Val-resin is incomplete, it may be necessary repeat coupling.

iv(a). Synthesis of Resin-Val-Succinate Methyl Ester

To a DMF solution of Resin-Val-succinic acid is added K₂CO₃ (1.0 eq.), followed by the addition of 20.0. eq. of dimethylcarbonate. After stirring at room temperature for 1 hour, the reaction mixture heated. Progress of the esterification reaction is monitored by HPLC, following cleavage of the resin bound Val-succinate dimer using 1% TFA/DCM. Alternatively, the allyl ester of Val-succinate is synthesized.

iv(b). Synthesis of Resin-Val-Succinate Allyl Ester

To a DCM solution of Resin-Val-succinic acid is added 1.1. eq. of triethyl amine followed by the addition of allyl bromide. After esterification is complete, the resin is washed with DMF (3×), followed by DCM (2×), then methanol (2×). The resin is dried wider vacuum prior to storage at a low temperature.

iv(c). Cleavage of Val-Succinate Methyl/Allyl Ester from Resin

The cold dry resin is brought to room temperature. A weighed aliquot of the resin is placed into a vial. To the resin is added DCM and the resulting slurry is stirred for about 45 min. to swell the resin. Following swelling, the DCM solution is withdrawn. The esterified product HO(O)C-Val-succinate methyl/allyl ester is cleaved from the resin using 1% TFA/DCM, (30 min).

### Synthesis of Formula Ia or Formula IIa Prodrugs

Scheme 1 illustrates the synthetic protocol for the manufacture of Formula Ia and Formula IIa prodrugs.

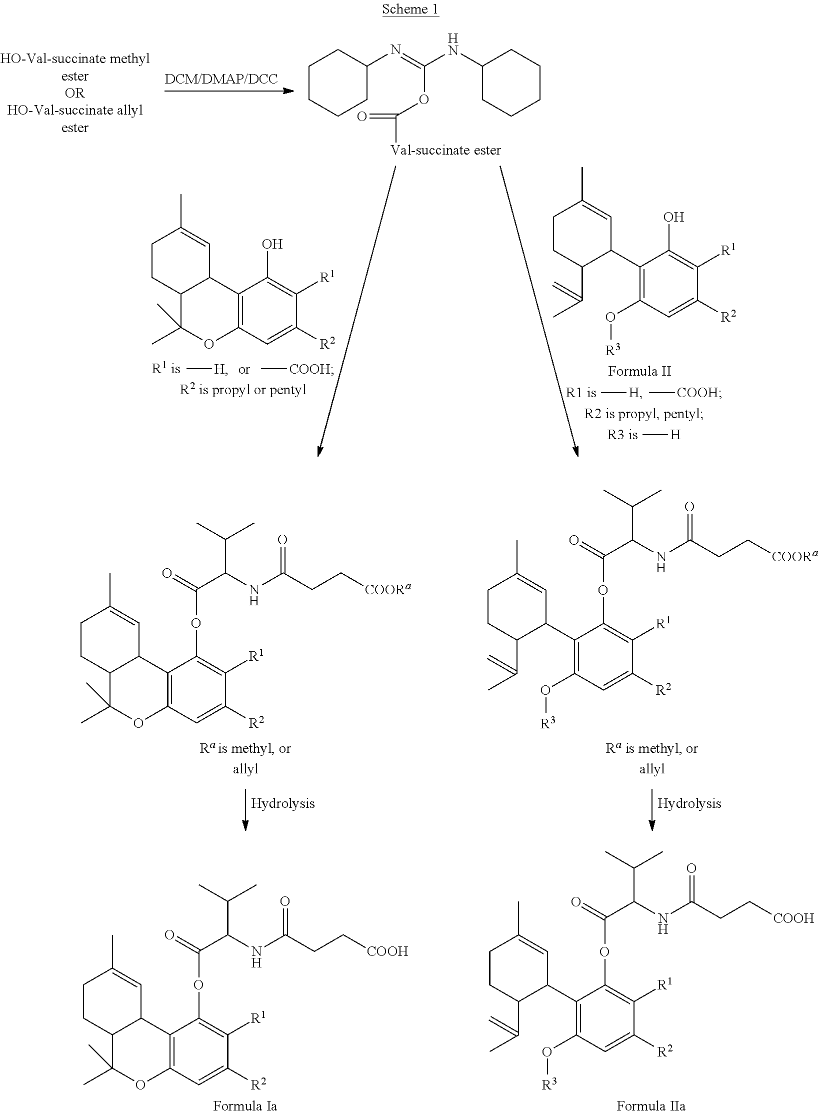

i(a). Coupling THCA to OH-Val-Succinate-Methyl Ester

Dicyclohexylcarbodiimide (DCC, 1.5 eq.) and DMAP are added to a dichloromethane solution of OH-Val-Succinate-Methyl ester. After stirring for about 30 minutes, dicyclohexyl urea formed as the by-product is filtered off. To the DCM solution of activated OH-Val-Succinate-Methyl ester is added drop-wise a DCM (or THF) solution of THCA. A catalytic amount of DMAP is added to the reaction mixture and reaction progress monitored by TLC or HPLC. Once coupling is complete, the reaction is quenched by the addition of citric acid (5% aq. solution), and the crude product extracted into the organic layer using DCM (3×). The combined DCM layers are washed with brine, dried over magnesium sulfate and concentrated prior to purification of the crude product by silica gel chromatography.

v(b). Hydrolysis of the Methyl Ester

De-esterification is accomplished by dissolving THCA-Val-succinate methyl ester in buffer at a pH of about 8.0-8.5.

v(c). Coupling THCA to OH-Val-Succinate-Allyl Ester

The synthetic protocol for coupling of OH-Val-Succinate-allyl ester is similar to the one described above for the coupling of OH-Val-Succinate-methyl ester to THCA.

### De-Esterification

After coupling, the allyl ester is de-protected using tetrakis(triphenylphosphine)Pd and phenyl silane using protocols well known in the peptide synthesis art. To a DCM/methanol solution of the allyl ester is added tetrakis(triphenylphosphine)Pd and phenyl silane. The reaction mixture is stirred under an inert atmosphere and progress of de-esterification is monitored periodically by HPLC or TLC. Following de-esterification, the catalyst is filtered off. Ammonium chloride is added to the reaction mixture and the pre-dominantly aqueous solution is extracted with ethyl acetate. The combined organic layers are dried over magnesium sulfate and the solvent is removed under vacuum to provide THCA-Val-succinic acid as a Formula Ia prodrug of the invention.

### Synthesis of Formula IVa or Formula Va Prodrugs

Scheme 2 illustrates an alternate strategy for manufacturing cannabinoid prodrugs of the invention.

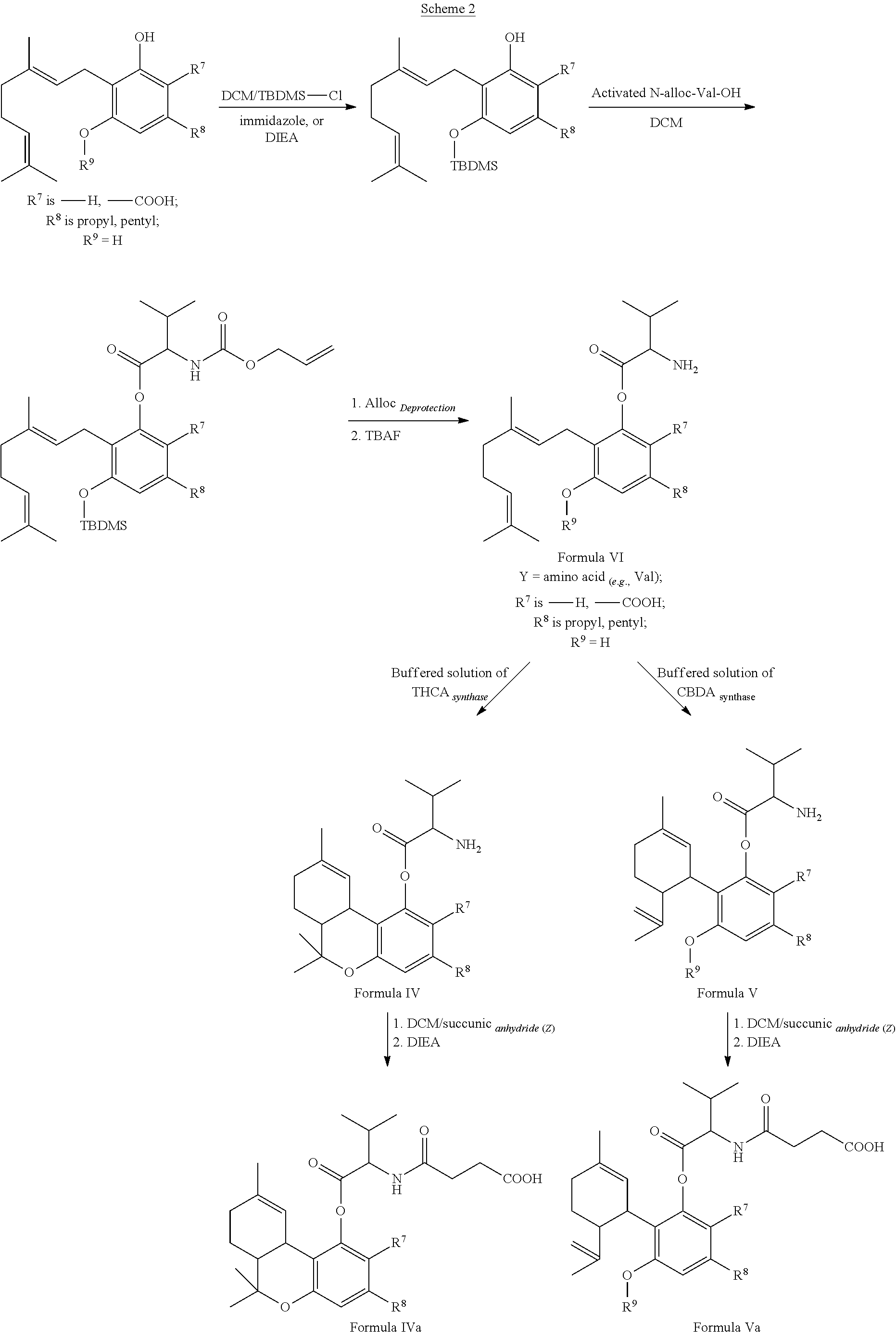

According to one embodiment, the formation of a Formula Va compound from a Formula V compound requires protection of the hydroxyl group (—OH, R⁹═—H) of the Formula V compound using hydroxyl protecting groups well known in the chemical synthetic art. Illustrative protecting groups include ten-butyldimethyl silyl (TBDMS), trimethyl silyl (TMS), acetyl, formyl, tetrahydropyranyl (THP), methoxymethyl (MOM), and trityl (Trt).

### Synthesis of 4-TBDMS-CBGA or 4-TBDMS-3-[3,7-dimethylocta-2,6-diene]-2-hydroxy-6-propyl benzoic acid

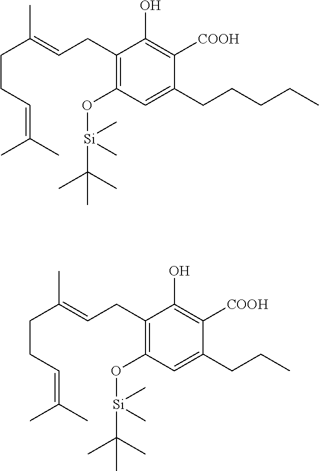 [SMILES: CCCCCc1cc(O[Si](C)(C)C(C)(C)C)c(C/C=C(\C)CCC=C(C)C)c(O)c1C(=O)O ; CCCc1cc(O[Si](C)(C)C(C)(C)C)c(C/C=C(\C)CCC=C(C)C)c(O)c1C(=O)O]

To a cold stirring solution of CBGA or 3-[3,7-dimethylocta-2,6-diene]-2,4-dihydroxy-6-propyl benzoic acid in DCM is added t-butyldimethylsilyl chloride (1.0 eq.) and imidazole. The reaction mixture is maintained under an atmosphere of argon and anhydrous solvent and reagents are used. TLC is used to monitor reaction progress. The reaction is quenched upon completion by the addition of brine. The organic layer was separated and dried using anhydrous magnesium sulfate prior to purification and use. If CBGA-ethyl ester is used as the starting material, the product can be hydrolyzed to the corresponding acid, if necessary, prior to enzyme-catalyzed synthesis of the cannabinoid prodrug.

### Synthesis of 2-((Allyloxy)carbonyl-Val-oxy)-4-(tert-butyldimethylsilyl)oxy-3-[3,7-dimethylocta-2,6-diene]-2-hydroxy-6-pentyl benzoic acid and 2-((Allyloxy)carbonyl-Val-oxy)-4-(tert-butyldimethylsilyl)oxy-3-[3,7-dimethylocta-2,6-diene]-2-hydroxy-6-propyl benzoic acid

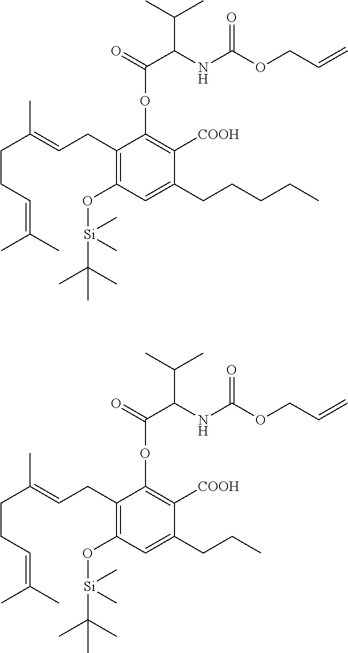 [SMILES: C=CCOC(=O)NC(C(=O)Oc1c(C/C=C(\C)CCC=C(C)C)c(O[Si](C)(C)C(C)(C)C)cc(CCCCC)c1C(=O)O)C(C)C ; C=CCOC(=O)NC(C(=O)Oc1c(C/C=C(\C)CCC=C(C)C)c(O[Si](C)(C)C(C)(C)C)cc(CCC)c1C(=O)O)C(C)C]

4-dimethylaminopyridine (DMAP) is added to a DCM solution of N-alloc-valine. To this solution, add N,N′-dicyclohexylcarbodiimide. After stirring at room temperature, a DCM solution of 4-TBDMS-CBGA or 4-TBDMS-3-[3,7-dimethylocta-2,6-diene]-2-hydroxy-6-propyl benzoic acid is added dropwise. The reaction mixture is stirred at room temperature overnight. The next day, the reaction mixture is filtered, and the filtrate is concentrated under reduced pressure prior to purification of the crude product by silica gel column chromatography.

### Removal of Alloc Protecting Group

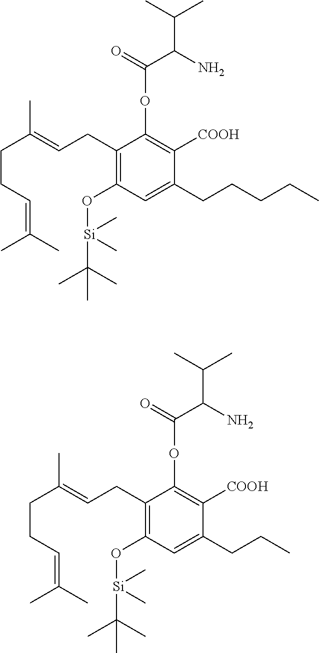 [SMILES: CCCCCc1cc(O[Si](C)(C)C(C)(C)C)c(C/C=C(\C)CCC=C(C)C)c(OC(=O)C(N)C(C)C)c1C(=O)O ; CCCc1cc(O[Si](C)(C)C(C)(C)C)c(C/C=C(\C)CCC=C(C)C)c(OC(=O)C(N)C(C)C)c1C(=O)O]

Removal of the alloc group is carried out using the catalyst tetrakis(triphenylphosphine)palladium in the presence of phenyl silane according to protocols well known in the peptide synthesis art. Briefly, the palladium catalyst and phenyl silane are added to a DCM/methanol solution of 2-((Allyloxy)carbonyl-valoxy)-4-(tert-butyldimethylsilyl)oxy-3-[3,7-dimethylocta-2,6-diene]-2-hydroxy-6-pentyl benzoic acid, or a DCM/methanol solution of 2-((Allyloxy)carbonyl-valoxy)-4-(tert-butyldimethylsilyl)oxy-3-[3,7-dimethylocta-2,6-diene]-2-hydroxy-6-propyl benzoic acid. The reaction mixture is stirred at room temperature and progress of the deprotection is monitored by HPLC. Following deprotection the reaction mixture will be filtered, then diluted with ammonium chloride and extracted using ethyl acetate (EtOAc). The combined organic layers are dried and the solvent removed to give 4-(tert-butyldimethylsilyl)oxy-3-[3,7-dimethylocta-2,6-diene]-2-hydroxy-6-pentyl-2-(valyloxy)benzoic acid and 4-(tert-butyldimethylsilyl)oxy-3-[3,7-dimethylocta-2,6-diene]-2-hydroxy-6-propyl-2-(valyloxy)benzoic acid respectively.

### Removal of TBDMS Group

### Synthesis of 3-[3,7-dimethylocta-2,6-diene]-4-hydroxy-6-pentyl-2-(valoxy)benzoic acid; and 3-[3,7-dimethylocta-2,6-diene]-4-hydroxy-6-propyl-2-(valoxy)benzoic acid

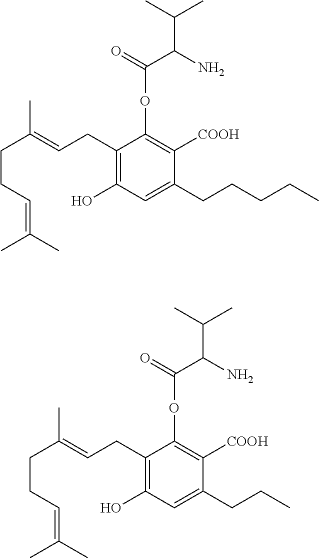 [SMILES: CCCCCc1cc(O)c(C/C=C(\C)CCC=C(C)C)c(OC(=O)C(N)C(C)C)c1C(=O)O ; CCCc1cc(O)c(C/C=C(\C)CCC=C(C)C)c(OC(=O)C(N)C(C)C)c1C(=O)O]

The TBDMS protecting group is removed by adding tetrabutylammonium fluoride or triethylamine trihydrofluoride to a DCM solution of 3-[3,7-dimethylocta-2,6-diene]-4-hydroxy-6-pentyl-2-(valyloxy)benzoic acid or a DCM solution of 3-[3,7-dimethylocta-2,6-diene]-4-hydroxy-6-propyl-2-(valyloxy)benzoic acid at −15° C. The reaction mixture is stirred at this temperature and TLC is used to monitor progress of deprotection. Following de-protection ethyl acetate (EtOAc is added to the reaction and the organic layer extracted (×3) using a dilute aqueous solution of sodium bicarbonate. The combined organic layers are dried and the solvent evaporated under reduced pressure prior to purification.

### Synthesis of a Formula IV or a Formula V Compound

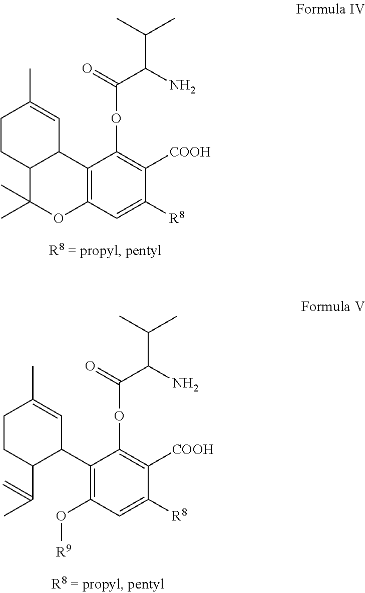

3-[3,7-dimethylocta-2,6-diene]-4-hydroxy-6-pentyl-2-(valyloxy)benzoic acid; or 3-[3,7-dimethylocta-2,6-diene]-4-hydroxy-6-propyl-2-(valyloxy)benzoic acid, prepared using the protocol described above, is added to a solution comprising cyclodextrin and buffer in a 1.0 ml eppendorf tube. After complete dissolution of the CBGA ester, the solution is incubated in a controlled temperature water bath maintained at 37° C., for at least 15 minutes before adding a known amount of a buffered solution of THCA synthase, or a known amount of a buffered solution of a CBDA synthase.

Following addition of the enzyme, a known aliquot of the reaction mixture, approximately 25 ul, is withdrawn at fixed intervals of time and the enzyme denatured by adding a fixed volume of ethanol. Following centrifugation of the precipitate, the ethanol layer is separated, dried and reconstituted in buffer. Progress of the reaction is followed spectrophotometrically or using HPLC.

The desired Formula IV and Formula V compounds are obtained by denaturing the enzyme using ethanol followed by evaporation of the ethanol layer to obtain crude Formula IV or V compounds.

### Synthesis of a Formula IVa or a Formula Va Compound

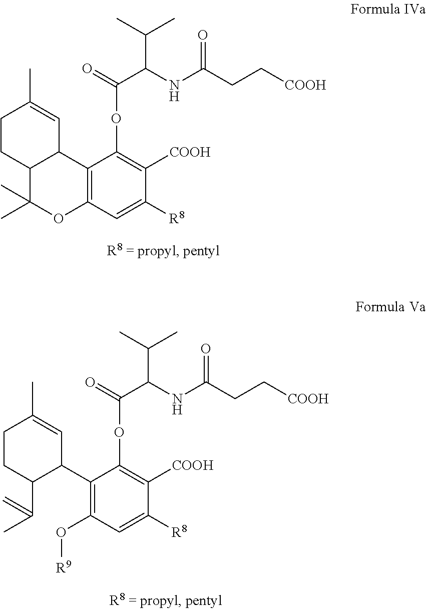

Succinic anhydride (1.1 eq.) is added to a DCM solution of an NH₂-Val-Formula IV compound or a NH₂-Val-Formula V compound (1.0 eq.). After stirring for a few minutes, DIEA or triethylamine (1.1 eq.) is added dropwise to the reaction mixture along with a catalytic amount of DMAP. The reaction mixture is stirred overnight and progress monitored by TLC. After the reaction is complete, the solvent is removed using a rotary evaporator. The crude product is dissolved in DCM and purified using silica gel column chromatography.

### Synthesis of Formula VIIa or Formula VIIIa Prodrugs

Scheme 3 illustrates yet another strategy for manufacturing cannabinoid prodrugs of the invention. According to this method, the 2-hydroxyl group of CBGA, or an analog of CBGA, is chemically modified to contain the prodrug moiety “—Y—Z”.

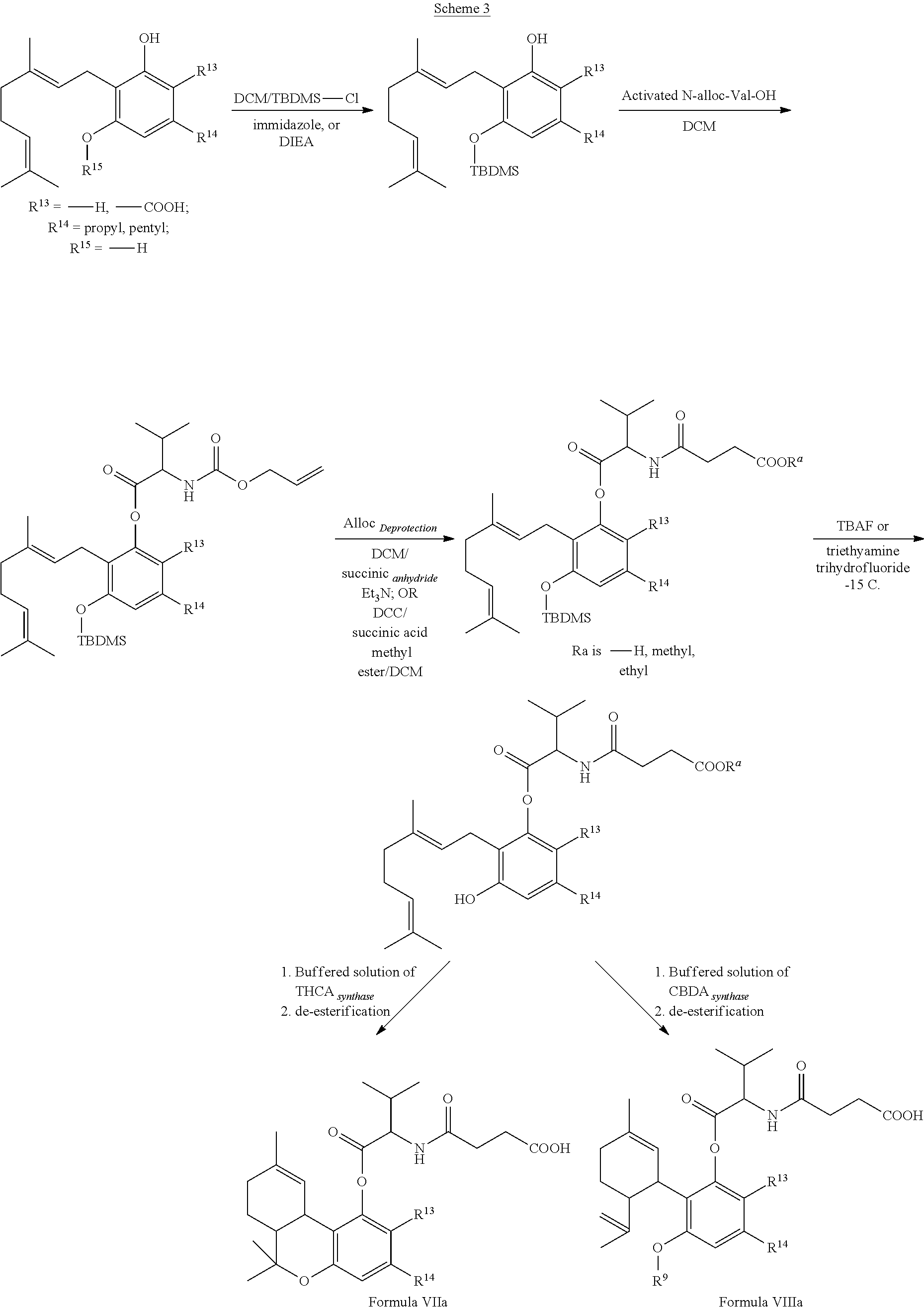

In one embodiment, CBGA is chemically modified to introduce exemplary prodrug moieties selected from the group consisting of Val-succinate, Ala-succinate, Lys-succinate, Phe-succinate, or Glu-succinate, thus producing a Formula IX compound which is the substrate for a cannabinoid enzyme.

According to another embodiment, the CBGA analog 3-[3,7-dimethylocta-2,6-diene]-2,4-dihydroxy-6-propylbenzoic acid is chemically modified to contain the prodrug moiety “—Y—Z”. The synthesis of these compounds proceeds by methods described herein. The first step is the protection of the 4-hydroxyl group of CBGA or 3-[3,7-dimethylocta-2,6-diene]-2,4-dihydroxy-6-propylbenzoic acid as the TBDMS ether.

In one embodiment, the 4-TBDMS-CBGA moiety is modified to include the desired prodrug moiety “—Y—Z” by (a) sequential addition of a “—Y” group and a “Z” group to the 2-hydroxyl group of 4-TBDMS-CBGA, or (b) by the conjugation of a —Y—Z synthon to the 2-hydroxyl group of 4-TBDMS-CBGA.

Methods to sequentially modify the 2-hydroxyl group of 4-TBDMS-CBGA as well as method for chemically modifying the 2-hydroxyl group of 4-TBDMS-CBGA using a synthon are described above. The Formula IX compound thus obtained is then used as the substrate of a suitable cannabinoid synthase enzyme.

Bio-enzymatic synthesis of the inventive Formula VIIa or Formula VIIIa prodrugs proceeds by dissolving the Formula IX substrate in a solution comprising cyclodextrin and buffer in a 1.0 ml eppendorf tube. This solution is incubated in a controlled temperature water bath maintained at 37° C., for at least 15 minutes before adding a known amount of a buffered solution of THCA synthase, or a known amount of a buffered solution of a CBDA synthase.

Following addition of the enzyme, a known aliquot of the reaction mixture, approximately 25 ul, is withdrawn at fixed intervals of time and the enzyme denatured by adding a fixed volume of ethanol. Following centrifugation of the precipitate, the ethanol layer is separated, dried and reconstituted in buffer. Progress of the enzyme catalyzed synthesis of a Formula VIIa or VIIIa prodrug is monitored spectrophotometrically or by HPLC.

### Purification of the Prodrugs

The cannabinoid prodrugs produced by bioenzymatic synthetic protocol described herein are purified by several analytical methods, including HPLC, size exclusion chromatography, and extraction into an organic solvent. The fractions corresponding to the desired prodrug product are pooled and lyophilized to dryness prior to use.

### E. Methods of Use

The naturally occurring cannabinoid tetrahydrocannabinol (THC), is gaining acceptance as a therapeutic for treating a wide range of medical conditions, including glaucoma, AIDS wasting, neuropathic pain, treatment of spasticity associated with multiple sclerosis, fibromyalgia and chemotherapy-induced nausea. THC is also effective in the treatment of allergies, inflammation, infection, epilepsy, depression, migraine, bipolar disorders, anxiety disorder, drug dependency and drug withdrawal syndromes.

The present invention provides prodrugs of natural cannabinoids as therapeutics for treating the above mentioned disorders. For instance, the inventive prodrugs when formulated for parenteral delivery are candidate therapeutics for alleviating pain. Such treatment is effected by administering a pharmaceutically acceptable formulation of the inventive prodrug alone or in combination with another pharmaceutical agent with known activity for reducing pain. The two pharmaceutical agents can be administered together or separately and the dose of each pharmaceutical agent is determined by the prescribing physician.

Prodrugs in accordance with the invention are also candidate therapeutics for treating inflammation. For instance, the inventive prodrugs can be administered to alleviate inflammation of the joints and associated pain in a subject with rheumatoid arthritis. The inventive prodrugs can be administered alone or in conjunction with a COX-inhibitor if necessary, at doses suitable for such treatment and deemed necessary by the prescribing physician.

We claim:

1. A method for producing a cannabinoid prodrug of Formula Ia or Formula IIa: 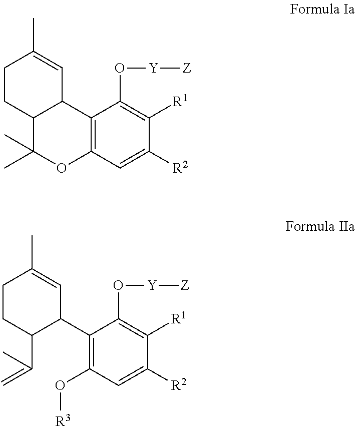
comprising the step of contacting a compound according to Formula I or Formula II 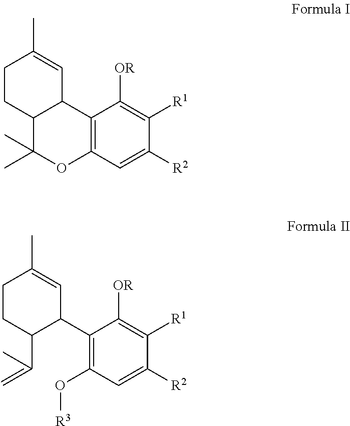
with an activated —Y—Z reagent to produce a prodrug according to Formula Ia or Formula IIa;
wherein,
R is —H;
R¹ is —H, —COOH, or —COO(C₁-C₅)alkyl;
R² is selected from the group consisting of (C₁-C₁₀)alkyl, (C₂-C₁₀)alkenyl, (C₂-C₁₀)alkynyl, (C₃-C₁₀)cycloalkyl, (C₃-C₁₀)cycloalkylalkylene, (C₃-C₁₀)aryl, and (C₃-C₁₀)arylalkylene;
R³ is —H, or (C₁-C₅)alkyl;
Z is selected from the group consisting of hemisuccinate, succinate, -oxalate, C(O)—CH₂—[OCH₂CH₂]ₙ—OR⁴, —C(O)—CH₂—[OCH₂CH₂]ₙ—NH₂, —C(O)[CH₂]ₙ—NR⁴R⁵, —C(O)O[CH₂]ₙ—NR⁴R⁵, —C(O)—NH—[CH₂]ₙ—NR⁴R⁵, —C(O)[CH₂]ₙ—N⁺(R⁴)(R⁵))(R⁶)X⁻, —C(O)O[CH₂]ₙ—N⁺(R⁴)(R⁵)(R⁶)X⁻, —C(O)—NH—[CH₂]ₙ—N⁺(R⁴)(R⁵))(R⁶)X⁻, and -oligosaccharide;
Y is selected from the group consisting of L-amino acid residue, a D-amino acid residue, a β-amino acid residue, a γ-amino acid residue, —C(O)—CH₂—[OCH₂CH₂]ₙ—O—, and —C(O)—CH₂—[OCH₂CH₂]ₙ—NH—; or —Y—Z is an oligosaccharide;
R⁴, R⁵, and R⁶ are each independently selected from the group consisting of —H, —OH, formyl, acetyl, pivaloyl, and (C₁-C₅)alkyl;
“n” is 1, 2, 3, 4, 5, or 6; and
“X” is a counter ion derived from a pharmaceutically acceptable acid.

2. The method of claim 1, wherein the compound according to Formula I or Formula II is obtained by contacting a compound of Formula III 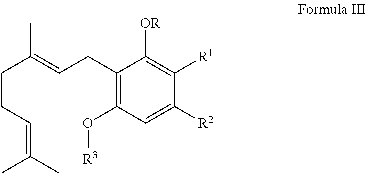
with a cannabinoid synthase, wherein substituents R, R¹, R², and R³ are as defined above.

3. The method of claim 2, wherein the compound of Formula III is contacted with the cannabinoid synthase in the presence of a solvent selected from the group consisting of water, phosphate buffer, citrate buffer, TRIS buffer, HEPES buffer, a mixture of water and a (C₁-C₅)alcohol, and a mixture of buffer and a (C₁-C₅)alcohol.

4. The method of claim 1, wherein Z is -hemisuccinate, -succinate, —C(O)—CH₂—[OCH₂CH₂]ₙ—OR⁴, or —C(O)—CH₂—[OCH₂CH₂]ₙ—NH₂.

5. The method of claim 4, wherein “Y” is an L-amino acid residue selected from the group consisting of glycine, valine, leucine, isoleucine, aspartic acid, glutamic acid, and lysine.

6. The method of claim 1, wherein —Y—Z is -valine-C(O)—CH₂—[OCH₂CH₂]ₙ—OR⁴, R⁴ is —H or methyl, and subscript “n” is 1, 2, 3, or 4.

7. The method of claim 1, wherein R¹ is —COOH, and R² is (C₁-C₁₀)alkyl.

8. The method of claim 7, wherein R² is propyl or pentyl.

9. The method of claim 2, wherein the cannabinoid synthase is selected from the group consisting of tetrahydrocannabivarin acid synthase (THCVA synthase), tetrahydrocannabinolic acid synthase (THCA synthase), cannabidiolic acid synthase, and cannabichromene acid synthase (CBCA synthase).

10. A method for producing a cannabinoid prodrug of Formula IVa or Formula Va: 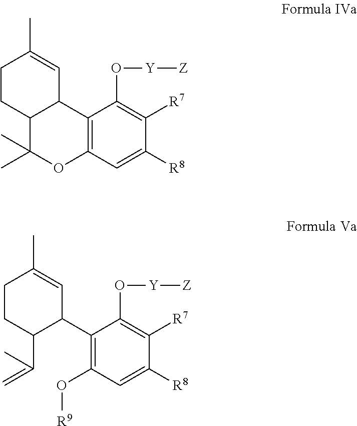
comprising
(a) contacting a compound of Formula VI: 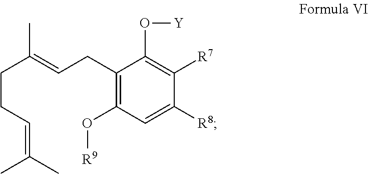
with a cannabinoid synthase to obtain a compound according to Formula IV or Formula V: 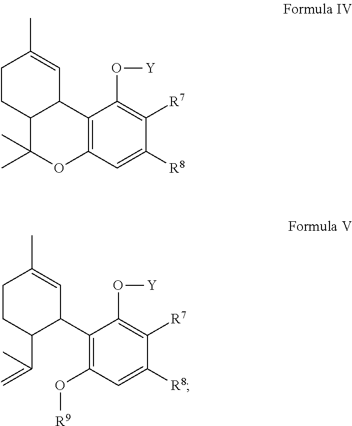
and
(b) contacting the compound according to Formula IV or Formula V with an activated —Z reagent to obtain the Formula IVa or Formula Va compound;
wherein
R⁷ is —H, —COOH, or —COO(C₁-C₅)alkyl;
R⁸ is selected from the group consisting of (C₁-C₁₀)alkyl, (C₂-C₁₀)alkenyl, (C₂-C₁₀)alkynyl, (C₃-C₁₀)cycloalkyl, (C₃-C₁₀)cycloalkylalkylene, (C₃-C₁₀)aryl, and (C₃-C₁₀)arylalkylene;
R⁹ is —H, or (C₁-C₅)alkyl;
Y is selected from the group consisting of L-amino acid residue, a D-amino acid residue, a β-amino acid residue, a γ-amino acid residue, and —C(O)—CH₂—[OCH₂CH₂]ₙ—NH—;
Z is selected from the group consisting of hemisuccinate, succinate, oxalate, —C(O)—CH₂—[OCH₂CH₂]ₙ—OR¹⁰, —C(O)—CH₂—[OCH₂CH₂]ₙ—NH₂, —C(O)[CH₂]ₙ—NR¹⁰R¹¹, —C(O)O[CH₂]ₙ—NR¹⁰R¹¹, —C(O)—NH—[CH₂]ₙ—NR¹⁰R¹¹, —C(O)[CH₂]ₙ—N⁺(R¹⁰)(R¹¹))(R¹²)X⁻, —C(O)O[CH₂]ₙ—N⁺(R¹⁰)(R¹¹)(R¹²)X⁻, and —C(O)—NH—[CH₂]ₙ—N⁺(R¹⁰)(R¹¹))(R¹²)X⁻,
R¹⁰, R¹¹, and R¹² are each independently selected from the group consisting of —H, —OH, formyl, acetyl, pivaloyl, and (C₁-C₅)alkyl;
“n” is 1, 2, 3, 4, 5, or 6; and
“X” is a counter ion derived from a pharmaceutically acceptable acid.

11. The method of claim 10, wherein the cannabinoid synthase is selected from the group consisting of tetrahydrocannabivarin acid synthase (THCVA synthase), tetrahydrocannabinolic acid synthase (THCA synthase), cannabidiolic acid synthase, and cannabichromene acid synthase (CBCA synthase).

12. The method of claim 10, wherein R⁷ is —COOH, and R⁸ is (C₁-C₁₀)alkyl.

13. The method of claim 12, wherein R⁸ is propyl or pentyl.

14. The method of claim 10, wherein —Y—Z in Formula IVa or Formula Va is selected from the group consisting of —Y-hemisuccinate, —Y-succinate, —Y—C(O)—CH₂—[OCH₂CH₂]ₙ—OR⁴, and —Y—C(O)—CH₂—[OCH₂CH₂]ₙ—NH₂.

15. The method of claim 14, wherein —Y—Z in Formula IVa or Formula Va is —Y-hemisuccinate.

16. A method for producing a cannabinoid prodrug of Formula VIIa or Formula VIIIa: 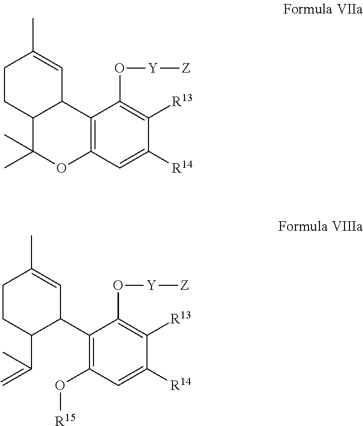
comprising
(a) contacting a compound of Formula IX: 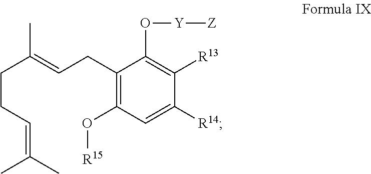
with a cannabinoid synthase to obtain the Formula VIIa or Formula VIIIa compound;
wherein
R¹³ is —H, —COOH, or —COO(C₁-C₅)alkyl;
R¹⁴ is selected from the group consisting of (C₁-C₁₀)alkyl, (C₂-C₁₀)alkenyl, (C₂-C₁₀)alkynyl, (C₃-C₁₀)cycloalkyl, (C₃-C₁₀)cycloalkylalkylene, (C₃-C₁₀)aryl, and (C₃-C₁₀)arylalkylene;
R¹⁵ is —H, or (C₁-C₅)alkyl;
—Y is selected from the group consisting of L-amino acid residue, a D-amino acid residue, a β-amino acid residue, a γ-amino acid residue, —C(O)—CH₂—[OCH₂CH₂]ₙ—O—, and —C(O)—CH₂—[OCH₂CH₂]ₙ—NH—;
—Z is selected from the group consisting of hemisuccinate, succinate, oxalate, —C(O)—CH₂—[OCH₂CH₂]ₙ—OR⁴, —C(O)—CH₂—[OCH₂CH₂]ₙ—NH₂, —C(O)[CH₂]ₙ—NR¹⁶R¹⁷, —C(O)O[CH₂]ₙ—NR¹⁶R¹⁷, —C(O)—NH—[CH₂]ₙ—NR¹⁶R¹⁷, —C(O)[CH₂]ₙ—N⁺(R¹⁶)(R¹⁷))(R¹⁸)X⁻, —C(O)O[CH₂]ₙ—N⁺(R¹⁶)(R¹⁷)(R¹⁸)X⁻, and —C(O)—NH—[CH₂]ₙ—N⁺(R¹⁶)(R¹⁷))(R¹⁸)X⁻, wherein
R¹⁶, R¹⁷, and R¹⁸ are each independently selected from the group consisting of —H, —OH, formyl, acetyl, pivaloyl, and (C₁-C₅)alkyl;
“n” is 1, 2, 3, 4, 5, or 6; and
“X” is a counter ion derived from a pharmaceutically acceptable acid.

17. The method of claim 16, wherein the cannabinoid synthase is selected from the group consisting of tetrahydrocannabivarin acid synthase (THCVA synthase), tetrahydrocannabinolic acid synthase (THCA synthase), cannabidiolic acid synthase, and cannabichromene acid synthase (CBCA synthase).

18. The method of claim 16, further comprising the step of contacting the Formula VIIa or Formula VIIIa compound with an oligosaccharide selected from the group consisting of mannose, N-acetyl glucosamine (GlcNAc), galactose, and sialic acid.

19. The method of claim 16, wherein —Y is an L-amino acid or a D-amino acid.

20. The method of claim 19, wherein —Y is an L-amino acid selected from the group consisting of valine, lysine, glutamic acid, and aspartic acid.
